(19) United States
(12) Patent Application Publication
(10) Pub. No.: US 2021/0205238 A1
(43) Pub. Date: Jul. 8, 2021

(54) STABILIZED INJECTABLE PHARMACEUTICAL COMPOSITION OF L-EPINEPHRINE

(71) Applicant: MERCK PATENT GMBH, DARMSTADT (DE)

(72) Inventors: Steffen AUGUSTIN, Reinbeck (DE); Thomas Artur Hendrik HERBST, Hemmingen (DE); Ulrich EMDE, Darmstadt (DE)

(73) Assignee: MERCK PATENT GMBH, DARMSTADT (DE)

(21) Appl. No.: 16/755,262

(22) PCT Filed: Oct. 8, 2018
(86) PCT No.: PCT/EP2018/077237

§ 371 Date: Apr. 10, 2020

(30) Foreign Application Priority Data
Oct. 10, 2017 (EP) 17195656.8

(51) Int. Cl. A61K 31/137; A61K 47/02; A61M 5/20; A61J 1/06

(52) U.S. Cl. CPC A61K 31/137; A61J 1/065; A61M 5/20; A61K 47/02

(57) ABSTRACT

The invention relates to injectable pharmaceutical compositions comprising epinephrine, an antioxidant selected from the group consisting of sodium metabisulfite, sodium sulfite and sodium bisulfite, tartrate, a tonicity regulating agent, EDTA or Na₂EDTA*2H₂O and pH 3.0-4.5.

The invention relates to injectable pharmaceutical compositions comprising epinephrine, an antioxidant selected from the group consisting of sodium metabisulfite, sodium sulfite and sodium bisulfite, tartrate, a tonicity regulating agent, EDTA or Na₂EDTA*2H₂O and pH 3.0-4.5.

### BACKGROUND OF THE INVENTION

Epinephrine, adrenaline, or (−)-3,4-Dihydroxy-[(methylamino)methyl]-benzyl-alcohol, is an endogenous adrenergic neurotransmitter synthesized and stored in the adrenal medulla. It is a polar compound characterized structurally by a catechol (a dihydroxybenzene) and an amine, and it is commonly available in a salt form. Epinephrine is water soluble and interacts in a variety of ways, depending on the type of receptor of target cells.

Epinephrine is one of the neural hormones responsible for the regulation of the heart, blood pressure, airway resistance, and energy metabolism. It is classified as a sympathomimetic drug, acting on both alpha and beta receptors. Epinephrine generates an inotropic effect, wherein it increases the heart rate, the force of contraction of the heart, narrows the blood vessels thus increasing blood pressure, reduces airway resistance to make it easier to breath, and raises blood glucose and blood fatty acids to supply the body energy during stress. Epinephrine is available in a variety of formulations suited for different clinical indications and routes of administration, for example by injection, by inhalation, or topically. Its uses include at least the following: combating low blood pressure during hemorrhagic or allergic shock; opening the airways during asthmatic attack; restricting the distribution of locally administered drugs such as local anesthetics; reducing nasal congestion; and/or performance aid in emergency situations.

Epinephrine can be prepared synthetically by one of several processes readily available to one in the art. One such process starts with 1,2-dihydroxybenzene that is converted successively to (chloroacetyl)catechol with chloroacetyl chloride, then to (methyl-aminoacetyl)catechol with methylamine and to racemic epinephrine by hydrogenation. The racemic form is resolved with D-tartaric acid to provide a white to nearly-white powder that is sensitive to light, air, heat, or alkaline conditions. Salts with acids are readily formed and provide some stability. The hydrochloride, sulphate, tartrate and bitartrate salts are known in the art.

Allergic emergencies, such as anaphylaxis, are a growing concern, given the increasing awareness of members of the public of their frequency and potential severity. Anaphylaxis is a sudden, severe, systemic allergic reaction that can be fatal, in many cases, if left untreated. Anaphylaxis can involve various areas of the body, such as the skin, respiratory tract, gastrointestinal tract, and cardiovascular system. Acute symptoms occur from within minutes to two hours after contact with the allergy-causing substance, but in rare instances onset may be delayed by as much as four hours. Contact with anaphylaxis-inducing agents and the severity of the resulting anaphylactic reaction are extremely unpredictable. Accordingly, allergists recommend that persons who have a personal or family history of anaphylaxis are always prepared to self-administer emergency treatment. Additionally, adults charged with caring for children who are at risk for anaphylaxis should also be prepared to administer anti-anaphylactic first aid.

The symptoms of anaphylaxis include one or more of the following, generally within 1 to about 15 minutes of exposure to the antigen: agitation, a feeling of uneasiness, flushing, palpitations, paresthesias, pruritus, throbbing in the ears, coughing, sneezing, urticaria, angioedema, difficulty breathing due to laryngeal edema or bronchospasm, nausea, vomiting, abdominal pain, diarrhea, shock, convulsions, incontinence, unresponsiveness and death. An anaphylactic reaction may include cardiovascular collapse, even in the absence of respiratory symptoms.

According to the Merck Manual, immediate treatment with epinephrine is imperative for the successful treatment of anaphylaxis (Merck Manual, 17. sup. Ed., 1053-1054 (1999)). The World Allergy Organization (WAO) Guidelines for the Assessment and Management of Anaphylaxis recommend a dose of 0.01 mg epinephrine/kg body weight to a maximum of 0.5 mg in adults and 0.3 mg in children; usually 1 mL administered as a 1:1000 dilution (1 mg/mL) or a 1:2000 dilution (0.5 mg/mL) of epinephrine in a suitable formulation. While the dose may be given manually, such as either subcutaneously or intramuscularly, for example, in recent years automatic injectors have become an accepted first aid means of delivering epinephrine. It is recommended that persons at risk of anaphylaxis, and persons responsible for children at risk for anaphylaxis, always maintain one or more automatic epinephrine injectors in a convenient place. It is further recommended that, if the symptoms of anaphylaxis persist after the first dose of epinephrine is injected, the patient should be treated with a second dose of epinephrine.

GB 425,678 discloses a process for producing a substantially stable anesthetic solution for local anesthesia containing an acid salt of an anesthetic, epinephrine or a physiological equivalent normally requiring an acid to keep it stable and an antioxidant, which comprises adjusting the pH value of the solution by a buffer so that the solution has a pH value within a range from approximately 5.7 up to approximately neutral. Sodium bisulfite is mentioned as antioxidant.

GB 930,452 and U.S. Pat. No. 3,149,035 disclose stable pharmaceutical solutions of a catechol amine comprising an aqueous solution of the catechol amine together with oxime, boric acid, and sodium bisulfite, said solutions having a pH of 6.5-6.8.

U.S. Pat. No. 3,966,905 discloses stabilized catechol amine solutions comprising a catechol amine, a polyvinylpyrrolidone, borate and a physiologically acceptable antioxidant selected from the group consisting of ascorbic acid, erythorbic acid, acetylcysteine, and thioglycerol, at a substantially neutral or mildly basic pH.

CA 981182 discloses the stabilization of L-epinephrine in a local anesthetic solution by using a combination of three specific antioxidants, i.e. bisulfite, ascorbic acid and thioglycerol, said solution comprising a local anesthetic selected from mepivacaine, bupivacaine and lidocaine, L-epinephrine, bisulfite, ascorbic acid and thioglycerol, and wherein said solution is of a pH of approximately 4.

DD-A1-150 694 discloses a formulation containing epinephrine hydrogen tartrate and sodium metabisulfite. WO 97/16196 and WO 98/2086 disclose formulations containing epinephrine and sodium metabisulfite. U.S. Pat. No. 4,734,438 discloses a formulation containing norepinephrine and sodium bisulfite.

US 2008/0269347 A1 discloses epinephrine formulations comprising epinephrine, EDTA, and at last one antioxidant, wherein the antioxidant is selected from the group consisting of cysteine, citric acid, thioglycerol, acetylcysteine, and a combination thereof. Sodium metabisufite as antioxidant is excluded since it has been associated with severe allergic reactions (see [0009]) and the authors do not comment the role of ETDA in the disclosed formulations.

WO 2014/202088 discloses stable epinephrine formulations with citric acid as antioxidant but with only very low epinephrine concentrations. The disclosed formulations may further comprise chelating agents as EDTA or EGTA but it is disclosed that the skilled person would know that citric acid may also be referred to as chelating agent.

WO 2014/127018, WO2014/127015 and WO2014/127020 disclose formulations comprising epinephrine, a complexing agent, e.g. sulfobutyl ether β-cyclodextrin or hydroxyl propyl β-cyclodextrin and a tonicity modifier in an aqueous solution. The complexing agent is contained to provide an inclusion complex with epinephrine and improved stability against thermal and/or oxidative degradations.

U.S. Pat. No. 9,119,876 B1 discloses a specific formulation containing 0.5 to 1.5 mg/mL epinephrine and/or its salts, 6 to 8 mg/mL of a tonicity regulating agent (e.g. sodium chloride), 2.8 to 3.8 mg/mL of a pH raising agent (e.g. tartaric acid and sodium hydroxide), 0.1 to 1.1 mg/mL of an antioxidant comprising at least sodium bisulfite and/or sodium metabilsulfite, 0.001 to 0.010 mg/mL of a pH lowering agent and 0.01 to 0.4 mg/mL of a transition metal complexing agent (e.g. EDTA). In column 22, lines 40-48 it is alleged, that the claimed pH range of most preferably 3.5-4.0 is responsible for the reduction of the D-epinephrine formation and in column 7, lines 24-50 it is disclosed that the transition metal complexing agent (e.g. EDTA) may inhibit the formation of degradants formed from the interaction of epinephrine, bisulfite and oxygen or inhibit the degradation of other components of the composition.

Examples for approved and commercially available epinephrine formulations for allergy emergency applications are the formulations for subcutaneous or intramuscular injection contained in the auto-injectors Epipen® (Fastjekt®), Emerade®, Jext®, Adrenaklick® and Auvi-Q®.

The Epipen® senior formulation contains 1.1 mg/mL epinephrine (Epipen® junior formulation 0.55 mg/mL epinephrine), 6.0 mg/mL sodium chloride, 1.67 mg/mL sodium metabisulfite (Na₂S₂O₅) and thus a ratio of epinephrine to sulfite (SO₃²⁻) equivalents of 0.34 (senior) or 0.17 (junior) (i.e. the molar ratio of the epinephrine compound and the antioxidant measured as sulfite equivalents (E:S)) and hydrochloric acid to pH 3.4. Adrenaklick® has a similar composition to Epipen®, but uses sodium bisulfite instead of sodium metabisulfite and includes chlorobutanol as a preservative. Auvi-Q® has a similar composition to Adrenaklick® but does not contain chlorobutanol.

EP 2437781 B1 and EP 2437782 B1 disclose liquid pharmaceutical compositions comprising epinephrine or salt thereof and an antioxidant selected from the group consisting of a bisulfite, a metabisulfite and a sulfite compound, wherein the molar ratio of epinephrine or the epinephrine salt to the antioxidant, measured as sulfite-equivalents (E:S), is in the range 0.70-1.30 or 1.31-2.20, respectively, and wherein the pH of said liquid composition is in the range of about 2.0-5.0. It is disclosed in [0056] that osmolality-adjusting agents (e.g. NaCl), pH adjusting agents (e.g. HCl or NaOH), chelating agents such as EDTA, carriers and other ingredients may be added. However, it is also disclosed in [0079] that at the disclosed metabisulfite levels, there seems to be no advantage using a chelating agent such as EDTA. Accordingly, the Jext® formulation contains 2 mg/mL epinephrine tartrate, 6 mg/mL sodium chloride, 0.57 mg/mL sodium metabisulfite (thus corresponding to an E:S ratio of 1.0) and hydrochloric acid to pH 3.4.

The Emerade® formulation contains 2 mg/mL epinephrine tartrate, 6 mg/mL sodium chloride, 0.5 mg/mL sodium metabisulfite (corresponding to an E:S ratio of 1.14), EDTA and hydrochloric acid to pH 3.4.

The L-configuration of epinephrine is 20 to 50 times more effective than the D-enantiomer. In pharmaceutical formulations L-epinephrine has been reported to degrade mainly by three different reactions: thermal or oxidative degradation, bisulfite addition and racemization (see Stepensky D. et al., J. Pharm. Sci, Vol. 93, No. 4, 969-980, 2004). Thus, shelf-life of epinephrine formulations is limited by the formation of degradants which are e.g. adrenochrome, epinephrine sulfonic acid (ESA) and D-epinephrine.

The modification or degradation of epinephrine is undesirable for several reasons. Modification of epinephrine results in loss of titer of the active ingredient, formation of compounds which may have undesirable physiological effects, and the appearance of a dark color, which makes the solution offensive and unmarketable. The initial loss of active compound due to auto-oxidation during the preparation and packaging of such a solution is substantial even though such procedures are carried out as nearly as practically possible in an inert atmosphere and such a solution must be stored temperature-controlled in order to decrease the rate of deterioration of the compound and thus prolong its shelf-life.

Epinephrine is destroyed readily in alkaline solutions by aldehydes, weak oxidizing agents, oxygen of the air and by auto-oxidation involving the formation of adrenaline-o-quinone, which in turn converts to adrenochrome. The rate of this reaction increases with pH and it has been found that the pH for maximum stability of epinephrine in solution is about 3-4.5. In alkaline solution and when exposed to air, light or elevated temperature epinephrine turns pink from oxidation to adrenochrome which is further degraded to adrenolutin and melanin or oxidation leads to the formation of polymers both with brown color.

In order to prevent oxidative degradation antioxidants such as cysteine, citric acid, thioglycerol, acetylcysteine, ascorbic acid, erythorbic acid, acetylcysteine, thioglycerol, bisulfite or sodium metabisulfite or complexing agents such as sulfobutyl ether β-cyclodextrin or hydroxyl propyl β-cyclodextrin are used in epinephrine formulations. In most commercially available epinephrine formulations sodium metabisulfite is used as antioxidant in order to prevent oxidative degradation of epinephrine but sodium metabisulfite or sodium bisulfite, has been associated with some severe allergic reactions.

In addition, sodium bisulfite can directly react with epinephrine to form a biologically inactive sulfonic acid derivative, epinephrine sulfonic acid (ESA). The safety and/or toxicity of ESA in commercial epinephrine products for anaphylactic treatment are still not well understood. In addition, the potency of epinephrine formulations also can be substantially degraded due to such reaction over product shelf-life.

Thus, due to the potential allergenic effect of sodium metabisulfite or sodium bisulfite and the formation of ESA the concentration of these antioxidants has to be kept relatively low and the skilled artesian would try to avoid high sodium bisulfite or sodium metabisulfite concentrations (corresponding to lower E:S ratios) than contained in the formulations of Epipen®, Adrenaclick® and AuviQ®, but would also favor sodium metabisulfite concentrations that are lower than those present in the formulations of Jext® and Emerade® (E:S ratios 1.0 and 1.14, respectively) or proposed in EP 2437781 B1 and EP 2437782 B1.

However, lowering the concentration of the antioxidants sodium metabisulfite or sodium bisulfite has the disadvantage of an increased oxidative degradation. Apparently, already at an E:S ratio of 1.14—as present in Emerade®—the antioxidant level is insufficient to guarantee long-term stability. In July 2017 Emerade® had to be recalled in Germany and the Netherlands due to discolorations and precipitation (https://www.deutsche-apotheker-zeitung.de/news/artikel/2017/07/04/wieder-ein-adrenalin-pen-rueckruf). According to this press release, the stability until the minimum durability of 30 months cannot be guaranteed and already after 24 months the Emerade® autoinjector does not meet the specification requirements anymore. New batches are sold with a minimum durability of only 18 months.

While oxidation can be prevented by addition of an antioxidant (like sodium metabisulfite) and bisulfite addition can be reduced by keeping the concentration of sulfite containing antioxidants as low as possible, strategies to prevent racemization have rarely been published. U.S. Pat. No. 9,119,876 B1 discloses that a slightly acidic pH range of 3.0-4.5 might be responsible for the reduction of the D-epinephrine formation. However, it was found in the experiments leading to the present invention that racemization of epinephrine in marketed formulations—like that of Epipen® with a pH of 3.4—is substantial and therefore should be prevented with respect to the fact that D-epinephrine is significantly less physiologically active than the L-form.

Thus, the aim of the present invention was to develop a formulation, which stabilizes L-epinephrine against the three major degradation pathways over shelf-life, maintains the adjusted pH and contains the lowest possible concentration of the potentially allergenic excipient sodium metabisulfite.

### SUMMARY OF THE INVENTION

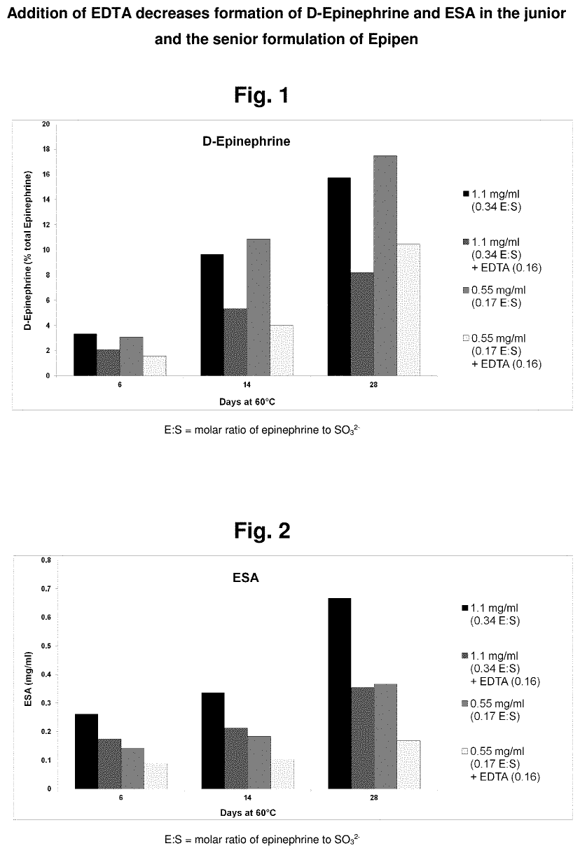

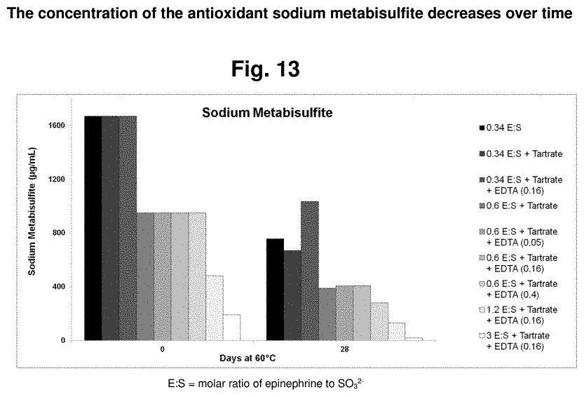

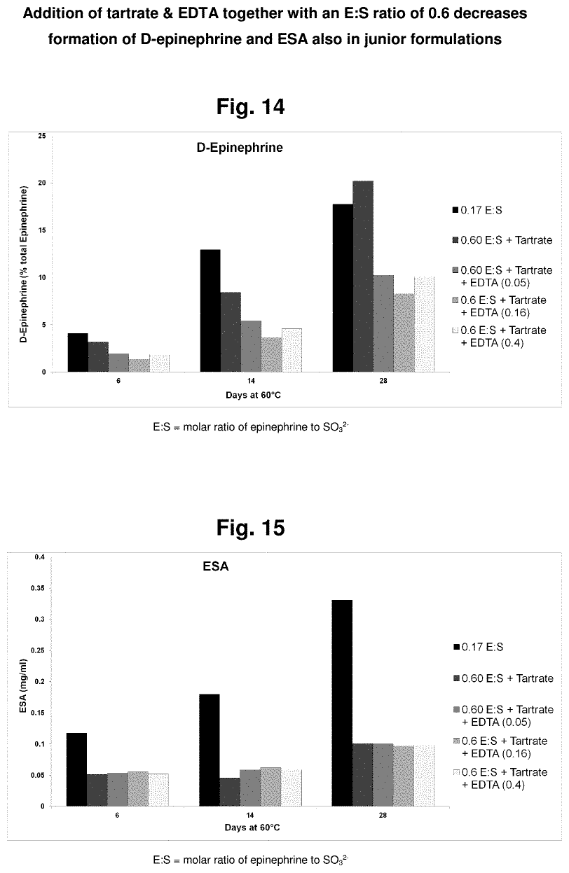

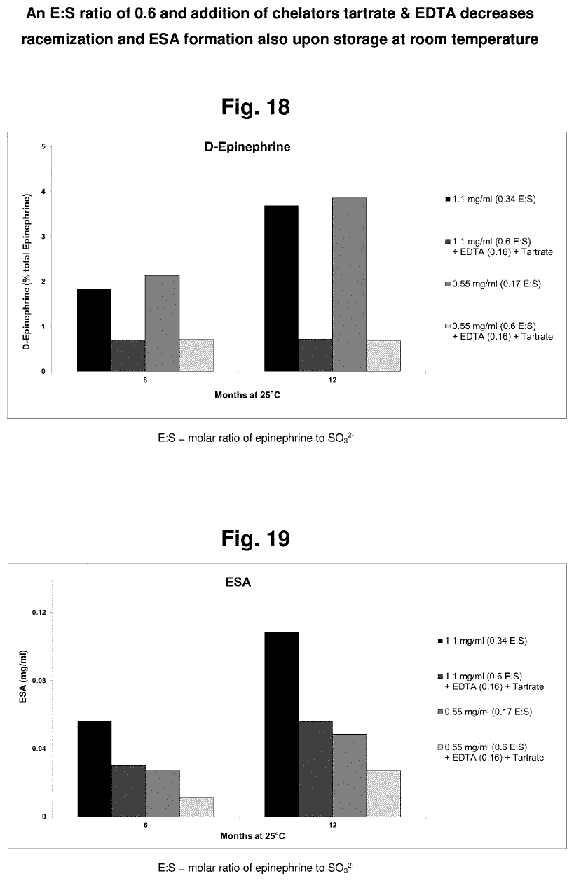

Surprisingly, it has been found, that the chelators tartrate and EDTA, in addition to reducing the ESA formation (bisulfite addition), reduce the formation of D-epinephrine (inhibit racemization) significantly over a wide sodium metabisulfite concentration range (see examples 5, 6, 7 and 8 and FIGS. 1-12). It has been also found that tartrate alone is not sufficient for long term reduction of D-epinephrine formation and that it is most efficient together with EDTA. In the presence of tartrate and EDTA minimal D-epinephrine formation was found at an E:S ratio of 0.6 and minimal ESA formation was observed at lower concentrations of sodium metabisulfite or E:S ratios of 3<1.2<0.6. However, since at E:S ratios ≥1.2 (or low concentrations of sodium metabisulfite) D-epinephrine formation increases significantly and oxidative damage at real time storage can occur as was shown by the recall of Emerade® in Germany in July 2017, the optimal E:S ratio considering D-epinephrine formation, ESA formation and oxidative degradation is 0.6. This also takes into account, that the concentration of the antioxidant sodium metabisulfite decreases drastically over time (see example 9 and FIG. 13), which show that at E:S=1.2 after 28 days at 60° C. already >70% and that at E:S=3 even >90% of the even low sodium metabisulfite starting content is degraded) thus leading to unacceptable low antioxidant concentrations after longer storage terms or at the end of shelf-life. The optimal E:S ratio of 0.6 found for senior formulations (1.1 mg/mL epinephrine) was also confirmed for junior formulations (0.55 mg/ml epinephrine) (see example 10 and FIGS. 14-17). These results are confirmed for both, the senior and the junior formulation upon long term storage over 6 and 12 months at room temperature (see example 11 and FIGS. 18-21).

Thus, surprisingly it has been found that by addition of the chelating agents EDTA and tartrate not only bisulfite addition is reduced (Milano E. A. et al., J. Parent. Sci. Techn. Vol. 36, No. 6, 232-236, 1982), but also the formation of D-epinephrine is significantly diminished. Furthermore, surprisingly it has been found that pharmaceutical epinephrine compositions containing EDTA in the range of 0.04 to 0.31 mg/mL, preferably 0.08-0.24 mg/mL, particularly preferred 0.09-0.16 mg/mL, most preferred 0.13 mg/mL or Na₂EDTA*2H₂O in the range of 0.05 to 0.4 mg/mL, preferably 0.1-0.3 mg/mL, particularly preferred 0.12-0.2 mg/mL, most preferred 0.16 mg/mL and having an E:S (molar ratio of the epinephrine compound and the antioxidant sodium metabisulfite, sodium sulfite or sodium bisulfite measured as sulfite equivalents (=sulfite ions, SO₃²⁻)) of 0.9-0.1, preferably 0.8-0.15, more preferred 0.7-0.3, particularly preferred 0.65-0.45, most preferred 0.6, for both, senior (6 mM epinephrine) and junior (3 mM epinephrine) formulations are the optimal pharmaceutical compositions of epinephrine in order to prevent all three different epinephrine degradation pathways (oxidation, bisulfite addition and racemization) and for keeping the concentration of the potentially allergenic excipient sodium metabisulfite as low as possible. Instead of EDTA, EGTA can be used analogously.

These pharmaceutical compositions correspond to epinephrine formulations with 3-9 mM, preferably 3.5-7 mM, more preferred 4.5-5.5 mM, particularly preferred 5 mM sodium metabisulfite (for senior) and formulations with 1.5-9 mM, preferably 1.7-7 mM, more preferred 2.3-3 mM, particularly preferred 2.5 mM sodium metabisulfite (for junior). Formulations with higher L-epinephrine: sulfite equivalent ratios (lower sodium metabisulfite concentration) are considered suboptimal due to the risk of oxidative damage as was shown by the recall of Emerade® in Germany in July 2017 and due to the observed significant decrease of the antioxidant sodium metabisulfite over time. Instead of sodium metabisulfite, sodium sulfite and sodium bisulfite can be used analogously. However, since sodium sulfite and sodium bisulfite merely contain one sulfite equivalent the corresponding concentrations are the epinephrine formulations of the present invention with 6-18 mM, preferably 7-14 mM, more preferred 9-11 mM, particularly preferred 10 mM sodium sulfite or sodium bisulfite (for senior) and formulations with 3-18 mM, preferably 3.4-14 mM, more preferred 4.6-6 mM, particularly preferred 5 mM sodium sulfite or sodium bisulfite (for junior).

Furthermore, in order to maintain the pH and to further stabilize the formulations, tartrate can be added as epinephrine tartrate or tartrate in a molar ratio of epinephrine to tartrate of 0.6-1.3, preferably 0.8-1.2, particularly preferred 1.0 for both senior and junior or the concentration of tartrate in the pharmaceutical compositions of the present invention is 2-8 mM, preferably 2-4 mM (junior formulation) or 4-8 mM (senior formulation), more preferred 2-4 mM (junior formulation) or 5-7 mM (senior formulation), particularly preferred 2.5-3.5 or 5.5-6.5 mM, most preferred 3 or 6 mM.

Furthermore, the concentration of epinephrine in the pharmaceutical compositions of the present invention is 2-8 mM, preferably 2-4 mM (junior formulation) or 4-8 mM (senior formulation), more preferred 2-4 mM (junior formulation) or 5-7 mM (senior formulation), particularly preferred 2.5-3.5 or 5.5-6.5 mM, most preferred 3 or 6 mM (junior or senior formulation, respectively).

Thus, an embodiment of the present invention are pharmaceutical compositions comprising epinephrine and an antioxidant selected from the group consisting of sodium metabisulfite, sodium sulfite and sodium bisulfite in a molar ratio of epinephrine to sulfite equivalents (E:S) of 0.9-0.1, 6-10 mg/mL of a tonicity regulating agent, 0.04-0.31 mg/mL EDTA or 0.05-0.4 mg/mL Na₂EDTA*2H₂O and pH 3-4.5, wherein the concentration of epinephrine is 2-8 mM.

A further embodiment of the present invention are pharmaceutical compositions comprising epinephrine and an antioxidant selected from the group consisting of sodium metabisulfite, sodium sulfite and sodium bisulfite in a molar ratio of epinephrine to sulfite equivalents of 0.9-0.1, tartrate in a molar ratio of epinephrine to tartrate of 0.6-1.3, 6-10 mg/mL of a tonicity regulating agent, 0.04-0.31 mg/mL EDTA or 0.05-0.4 mg/mL Na₂EDTA*2H₂O and pH 3-4.5, wherein the concentration of epinephrine is 2-8 mM.

Another embodiment of the present invention are pharmaceutical compositions comprising epinephrine and sodium metabisulfite in a molar ratio of epinephrine to sulfite equivalents of 0.9-0.1, tartrate in a molar ratio of epinephrine to tartrate of 0.6-1.3, 6-10 mg/mL of a tonicity regulating agent, 0.04-0.31 mg/mL EDTA or 0.05-0.4 mg/mL Na₂EDTA*2H₂O and pH 3-4.5, wherein the concentration of epinephrine is 2-8 mM.

A preferred embodiment of the present invention are pharmaceutical compositions comprising epinephrine and an antioxidant selected from the group consisting of sodium metabisulfite, sodium sulfite and sodium bisulfite in a molar ratio of epinephrine to sulfite equivalents of 0.8-0.15, 6-10 mg/mL of a tonicity regulating agent, 0.04-0.31 mg/mL EDTA or 0.05-0.4 mg/mL Na₂EDTA*2H₂O and pH 3-4.5, wherein the concentration of epinephrine is 2-4 or 4-8 mM for junior and senior formulations, respectively.

A further preferred embodiment of the present invention are pharmaceutical compositions comprising epinephrine and an antioxidant selected from the group consisting of sodium metabisulfite, sodium sulfite and sodium bisulfite in a molar ratio of epinephrine to sulfite equivalents of 0.8-0.15, tartrate in a molar ratio of epinephrine to tartrate of 0.8-1.2, 6-10 mg/mL of a tonicity regulating agent, 0.04-0.31 mg/mL EDTA or 0.05-0.4 mg/mL Na₂EDTA*2H₂O and pH 3-4.5, wherein the concentration of epinephrine is 2-4 or 4-8 mM for junior and senior formulations, respectively.

Another preferred embodiment of the present invention are pharmaceutical compositions comprising epinephrine and sodium metabisulfite in a molar ratio of epinephrine to sulfite equivalents of 0.8-0.15, tartrate in a molar ratio of epinephrine to tartrate of 0.8-1.2, 6-10 mg/mL of a tonicity regulating agent, 0.04-0.31 mg/mL EDTA or 0.05-0.4 mg/mL Na₂EDTA*2H₂O and pH 3-4.5, wherein the concentration of epinephrine is 2-4 or 4-8 mM for junior and senior formulations, respectively.

A more preferred embodiment of the present invention are pharmaceutical compositions comprising epinephrine and an antioxidant selected from the group consisting of sodium metabisulfite, sodium sulfite and sodium bisulfite in a molar ratio of epinephrine to sulfite equivalents of 0.7-0.2, 8-9.5 mg/mL of a tonicity regulating agent, 0.08-0.24 mg/mL EDTA or 0.1-0.3 mg/mL Na₂EDTA*2H₂O and pH 3.3-4.2, wherein the concentration of epinephrine is 2-4 or 5-7 mM.

Another more preferred embodiment of the present invention are pharmaceutical compositions comprising epinephrine and an antioxidant selected from the group consisting of sodium metabisulfite, sodium sulfite and sodium bisulfite in a molar ratio of epinephrine to sulfite equivalents of 0.7-0.2, tartrate in a molar ratio of epinephrine to tartrate of 0.8-1.2, 8-9.5 mg/mL of a tonicity regulating agent, 0.08-0.24 mg/mL EDTA or 0.1-0.3 mg/mL Na₂EDTA*2H₂O and pH 3.3-4.2, wherein the concentration of epinephrine is 2-4 or 5-7 mM.

Another more preferred embodiment of the present invention are pharmaceutical compositions comprising epinephrine and sodium metabisulfite in a molar ratio of epinephrine to sulfite equivalents of 0.7-0.2, tartrate in a molar ratio of epinephrine to tartrate of 0.8-1.2, 8-9.5 mg/mL of a tonicity regulating agent, 0.08-0.24 mg/mL EDTA or 0.1-0.3 mg/mL Na₂EDTA*2H₂O and pH 3.3-4.2, wherein the concentration of epinephrine is 2-4 or 5-7 mM.

Another more preferred embodiment of the present invention are pharmaceutical compositions comprising epinephrine and an antioxidant selected from the group consisting of sodium metabisulfite, sodium sulfite and sodium bisulfite in a molar ratio of epinephrine to sulfite equivalents of 0.65-0.45, 8-9.5 mg/mL NaCl, 0.09-0.16 mg/mL EDTA or 0.12-0.2 mg/mL Na₂EDTA*2H₂O and pH 3.3-4.2, wherein the concentration of epinephrine is 2.5-3.5 or 5.5-6.5 mM.

Another more preferred embodiment of the present invention are pharmaceutical compositions comprising epinephrine and an antioxidant selected from the group consisting of sodium metabisulfite, sodium sulfite and sodium bisulfite in a molar ratio of epinephrine to sulfite equivalents of 0.65-0.45, tartrate in a molar ratio of epinephrine to tartrate of 0.8-1.2, 8-9.5 mg/mL NaCl, 0.09-0.16 mg/mL EDTA or 0.12-0.2 mg/mL Na₂EDTA*2H₂O and pH 3.3-4.2, wherein the concentration of epinephrine is 2.5-3.5 or 5.5-6.5 mM.

Another more preferred embodiment of the present invention are pharmaceutical compositions comprising epinephrine and sodium metabisulfite in a molar ratio of epinephrine to sulfite equivalents of 0.65-0.45, tartrate in a molar ratio of epinephrine to tartrate of 0.8-1.2, 8-9.5 mg/mL NaCl, 0.09-0.16 mg/mL EDTA or 0.12-0.2 mg/mL Na₂EDTA*2H₂O and pH 3.3-4.2, wherein the concentration of epinephrine is 2.5-3.5 or 5.5-6.5 mM.

A particularly embodiment of the present invention are pharmaceutical compositions comprising epinephrine and an antioxidant selected from the group consisting of sodium metabisulfite, sodium sulfite and sodium bisulfite in a molar ratio of epinephrine to sulfite equivalents of 0.6, 8-9.5 mg/mL NaCl, 0.09-0.16 mg/mL EDTA or 0.12-0.2 mg/mL Na₂EDTA*2H₂O and pH 3.3-4.2, wherein the concentration of epinephrine is 2.5-3.5 or 5.5-6.5 mM.

A further particularly embodiment of the present invention are pharmaceutical compositions comprising epinephrine and an antioxidant selected from the group consisting of sodium metabisulfite, sodium sulfite and sodium bisulfite in a molar ratio of epinephrine to sulfite equivalents of 0.6, tartrate in a molar ratio of epinephrine to tartrate of 0.8-1.2, 8-9.5 mg/mL NaCl, 0.09-0.16 mg/mL EDTA or 0.12-0.2 mg/mL Na₂EDTA*2H₂O and pH 3.3-4.2, wherein the concentration of epinephrine is 2.5-3.5 or 5.5-6.5 mM.

Another particularly embodiment of the present invention are pharmaceutical compositions comprising epinephrine and sodium metabisulfite in a molar ratio of epinephrine to sulfite equivalents of 0.6, tartrate in a molar ratio of epinephrine to tartrate of 0.8-1.2, 8-9.5 mg/mL NaCl, 0.09-0.16 mg/mL EDTA or 0.12-0.2 mg/mL Na₂EDTA*2H₂O and pH 3.3-4.2, wherein the concentration of epinephrine is 2.5-3.5 or 5.5-6.5 mM.

Another particularly embodiment of the present invention are pharmaceutical compositions comprising epinephrine and sodium metabisulfite in a molar ratio of epinephrine to sulfite equivalents 0.6, tartrate in a molar ratio of epinephrine to tartrate of 1.0, 8-9.5 mg/mL NaCl, 0.13 mg/mL EDTA or 0.16 mg/mL Na₂EDTA*2H₂O and pH 3.7-4.2, wherein the concentration of epinephrine is 2.5-3.5 or 5.5-6.5 mM.

Another particularly embodiment of the present invention are pharmaceutical compositions comprising epinephrine and sodium metabisulfite in a molar ratio of epinephrine to sulfite equivalents 0.6, tartrate in a molar ratio of epinephrine to tartrate of 1.0, 8-9.5 mg/mL NaCl, 0.13 mg/mL EDTA or 0.16 mg/mL Na₂EDTA*2H₂O and pH 3.8-4.0, wherein the concentration of epinephrine is 3 or 6 mM.

Another particularly embodiment of the present invention are pharmaceutical compositions comprising epinephrine and sodium metabisulfite in a molar ratio of epinephrine to sulfite equivalents 0.6, tartrate in a molar ratio of epinephrine to tartrate of 1.0, 8.2-9.2 mg/mL NaCl, 0.13 mg/mL EDTA or 0.16 mg/mL Na₂EDTA*2H₂O and pH 3.9, wherein the concentration of epinephrine is 3 or 6 mM.

A further embodiment of the present invention are pharmaceutical compositions used as senior formulation for adults comprising 4-8 mM epinephrine, an antioxidant selected from the group consisting of 3-9 mM sodium metabisulfite, 6-18 mM sodium sulfite and 6-18 mM sodium bisulfite, 6-10 mg/mL of a tonicity regulating agent, 0.04-0.31 mg/mL EDTA or 0.05-0.4 mg/mL Na₂EDTA*2H₂O and pH 3-4.5.

A further embodiment of the present invention are pharmaceutical compositions used as senior formulation for adults comprising 4-8 mM epinephrine, an antioxidant selected from the group consisting of 3-9 mM sodium metabisulfite, 6-18 mM sodium sulfite and 6-18 mM sodium bisulfite, 4-8 mM tartrate, 6-10 mg/mL of a tonicity regulating agent, 0.04-0.31 mg/mL EDTA or 0.05-0.4 mg/mL Na₂EDTA*2H₂O and pH 3-4.5.

A further embodiment of the present invention are pharmaceutical compositions used as senior formulation for adults comprising 4-8 mM epinephrine, 3-9 mM sodium metabisulfite, 4-8 mM tartrate, 6-10 mg/mL of a tonicity regulating agent, 0.04-0.31 mg/mL EDTA or 0.05-0.4 mg/mL Na₂EDTA*2H₂O and pH 3-4.5.

A preferred embodiment of the present invention are pharmaceutical compositions used as senior formulation for adults comprising 5-7 mM epinephrine, an antioxidant selected from the group consisting of 3.5-7 mM sodium metabisulfite, 7-14 mM sodium sulfite and 7-14 mM sodium bisulfite, 8-9 mg/mL of a tonicity regulating agent, 0.08-0.24 mg/mL EDTA or 0.1-0.3 mg/mL EDTA and pH 3.3-4.2.

A further preferred embodiment of the present invention are pharmaceutical compositions used as senior formulation for adults comprising 5-7 mM epinephrine, an antioxidant selected from the group consisting of 3.5-7 mM sodium metabisulfite, 7-14 mM sodium sulfite and 7-14 mM sodium bisulfite, 5-7 mM tartrate, 8-9 mg/mL of a tonicity regulating agent, 0.08-0.24 mg/mL EDTA or 0.1-0.3 mg/mL EDTA and pH 3.3-4.2.

Another preferred embodiment of the present invention are pharmaceutical compositions used as senior formulation for adults comprising 5-7 mM epinephrine, 3.5-7 mM sodium metabisulfite, 5-7 mM tartrate, 8-9 mg/mL of a tonicity regulating agent, 0.08-0.24 mg/mL EDTA or 0.1-0.3 mg/mL EDTA and pH 3.3-4.2.

A more preferred embodiment of the present invention are pharmaceutical compositions used as senior formulation for adults comprising 5.5-6.5 mM epinephrine, an antioxidant selected from the group consisting of 4.5-5.5 mM sodium metabisulfite, 9-11 mM sodium sulfite and 9-11 mM sodium bisulfite, 8-9 mg/mL NaCl, 0.09-0.16 mg/mL EDTA or 0.12-0.2 mg/mL Na₂EDTA*2H₂O and pH 3.3-4.2.

Another more preferred embodiment of the present invention are pharmaceutical compositions used as senior formulation for adults comprising 5.5-6.5 mM epinephrine, an antioxidant selected from the group consisting of 4.5-5.5 mM sodium metabisulfite, 9-11 mM sodium sulfite and 9-11 mM sodium bisulfite, 5.5-6.5 mM tartrate, 8-9 mg/mL NaCl, 0.09-0.16 mg/mL EDTA or 0.12-0.2 mg/mL Na₂EDTA*2H₂O and pH 3.3-4.2.

Another more preferred embodiment of the present invention are pharmaceutical compositions used as senior formulation for adults comprising 5.5-6.5 mM epinephrine, 4.5-5.5 mM sodium metabisulfite, 5.5-6.5 mM tartrate, 8-9 mg/mL NaCl, 0.09-0.16 mg/mL EDTA or 0.12-0.2 mg/mL Na₂EDTA*2H₂O and pH 3.3-4.2.

A particularly embodiment of the present invention are pharmaceutical compositions used as senior formulation for adults comprising 6 mM epinephrine, an antioxidant selected from the group consisting of 5.0 mM sodium metabisulfite, 10 mM sodium sulfite and 10 mM sodium bisulfite, 8-9 mg/mL NaCl, 0.13 mg/mL EDTA or 0.16 mg/mL Na₂EDTA*2H₂O and pH 3.7-4.2.

Another particularly embodiment of the present invention are pharmaceutical compositions used as senior formulation for adults comprising 6 mM epinephrine, an antioxidant selected from the group consisting of 5.0 mM sodium metabisulfite, 10 mM sodium sulfite and 10 mM sodium bisulfite, 6 mM tartrate, 8-9 mg/mL NaCl, 0.13 mg/mL EDTA or 0.16 mg/mL Na₂EDTA*2H₂O and pH 3.7-4.2.

Another particularly embodiment of the present invention are pharmaceutical compositions used as senior formulation for adults comprising 6 mM epinephrine, 5.0 mM sodium metabisulfite, 6 mM tartrate, 8-9 mg/mL NaCl, 0.13 mg/mL EDTA or 0.16 mg/mL Na₂EDTA*2H₂O and pH 3.7-4.2.

Another particularly embodiment of the present invention are pharmaceutical compositions used as senior formulation for adults comprising 6 mM epinephrine, 5.0 mM sodium metabisulfite, 6 mM tartrate, 8.4 mg/mL NaCl, 0.13 mg/mL EDTA or 0.16 mg/mL Na₂EDTA*2H₂O and pH 3.9.

Another particularly embodiment of the present invention is pharmaceutical composition used as senior formulation for adults comprising 2 mg/mL epinephrine tartrate or 1.1 mg/mL epinephrine base and 0.9 mg/mL tartrate, 0.95 mg/mL sodium metabisulfite, 8.4 mg/mL NaCl, 0.13 mg/mL EDTA or 0.16 mg/mL Na₂EDTA*2H₂O and pH 3.9.

Another embodiment of the present invention are pharmaceutical compositions used as junior formulation for children comprising 2-4 mM epinephrine, an antioxidant selected from the group consisting of 1.5-9 mM sodium metabisulfite, 3-18 mM sodium sulfite and 3-18 mM sodium bisulfite, 6-10 mg/mL of a tonicity regulating agent, 0.04-0.31 mg/mL EDTA or 0.05-0.4 mg/mL Na₂EDTA*2H₂O and pH 3-4.5.

Another embodiment of the present invention are pharmaceutical compositions used as junior formulation for children comprising 2-4 mM epinephrine, an antioxidant selected from the group consisting of 1.5-9 mM sodium metabisulfite, 3-18 mM sodium sulfite and 3-18 mM sodium bisulfite, 2-4 mM tartrate, 6-10 mg/mL of a tonicity regulating agent, 0.04-0.31 mg/mL EDTA or 0.05-0.4 mg/mL Na₂EDTA*2H₂O and pH 3-4.5.

Another embodiment of the present invention are pharmaceutical compositions used as junior formulation for children comprising 2-4 mM epinephrine, 1.5-9 mM sodium metabisulfite, 2-4 mM tartrate, 6-10 mg/mL of a tonicity regulating agent, 0.04-0.31 mg/mL EDTA or 0.05-0.4 mg/mL Na₂EDTA*2H₂O and pH 3-4.5.

A preferred embodiment of the present invention are pharmaceutical compositions used as junior formulation for children comprising 2-4 mM epinephrine, an antioxidant selected from the group consisting of 1.7-7 mM sodium metabisulfite, 3.4-14 mM sodium sulfite and 3.4-14 mM sodium bisulfite, 8.5-9.5 mg/mL of a tonicity regulating agent, 0.08-0.24 mg/mL EDTA or 0.1-0.3 mg/mL Na₂EDTA*2H₂O and pH 3.3-4.2.

Another preferred embodiment of the present invention are pharmaceutical compositions used as junior formulation for children comprising 2-4 mM epinephrine, an antioxidant selected from the group consisting of 1.7-7 mM sodium metabisulfite, 3.4-14 mM sodium sulfite and 3.4-14 mM sodium bisulfite, 2-4 mM tartrate, 8.5-9.5 mg/mL of a tonicity regulating agent, 0.08-0.24 mg/mL EDTA or 0.1-0.3 mg/mL Na₂EDTA*2H₂O and pH 3.3-4.2.

Another preferred embodiment of the present invention are pharmaceutical compositions used as junior formulation for children comprising 2-4 mM epinephrine, 1.7-7 mM sodium metabisulfite, 2-4 mM tartrate, 8.5-9.5 mg/mL of a tonicity regulating agent, 0.08-0.24 mg/mL EDTA or 0.1-0.3 mg/mL Na₂EDTA*2H₂O and pH 3.3-4.2.

A more preferred embodiment of the present invention are pharmaceutical compositions used as junior formulation for children comprising 2.5-3.5 mM epinephrine, an antioxidant selected from the group consisting of 2.3-3 mM sodium metabisulfite, 4.6-6 mM sodium sulfite and 4.6-6 mM sodium bisulfite, 8.5-9.5 mg/mL NaCl, 0.09-0.16 mg/mL EDTA or 0.12-0.2 mg/mL Na₂EDTA*2H₂O and pH 3.3-4.2.

Another more preferred embodiment of the present invention are pharmaceutical compositions used as junior formulation for children comprising 2.5-3.5 mM epinephrine, an antioxidant selected from the group consisting of 2.3-3 mM sodium metabisulfite, 4.6-6 mM sodium sulfite and 4.6-6 mM sodium bisulfite, 2.5-3.5 mM tartrate, 8.5-9.5 mg/mL NaCl, 0.09-0.16 mg/mL EDTA or 0.12-0.2 mg/mL Na₂EDTA*2H₂O and pH 3.3-4.2.

Another more preferred embodiment of the present invention are pharmaceutical compositions used as junior formulation for children comprising 2.5-3.5 mM epinephrine, 2.3-3 mM sodium metabisulfite, 2.5-3.5 mM tartrate, 8.5-9.5 mg/mL NaCl, 0.09-0.16 mg/mL EDTA or 0.12-0.2 mg/mL Na₂EDTA*2H₂O and pH 3.3-4.2.

A particularly preferred embodiment of the present invention are pharmaceutical compositions used as junior formulation for children comprising 3 mM epinephrine, an antioxidant selected from the group consisting of 2.5 mM sodium metabisulfite, 5 mM sodium sulfite and 5 mM sodium bisulfite, 8.5-9.5 mg/mL NaCl, 0.13 mg/mL EDTA or 0.16 mg/mL Na₂EDTA*2H₂O and pH 3.7-4.2.

A particularly preferred embodiment of the present invention are pharmaceutical compositions used as junior formulation for children comprising 3 mM epinephrine, an antioxidant selected from the group consisting of 2.5 mM sodium metabisulfite, 5 mM sodium sulfite and 5 mM sodium bisulfite, 3 mM tartrate, 8.5-9.5 mg/mL NaCl, 0.13 mg/mL EDTA or 0.16 mg/mL Na₂EDTA*2H₂O and pH 3.7-4.2.

A particularly preferred embodiment of the present invention are pharmaceutical compositions used as junior formulation for children comprising 3 mM epinephrine, 2.5 mM sodium metabisulfite, 3 mM tartrate, 8.5-9.5 mg/mL NaCl, 0.13 mg/mL EDTA or 0.16 mg/mL Na₂EDTA*2H₂O and pH 3.7-4.2.

A particularly preferred embodiment of the present invention are pharmaceutical compositions used as junior formulation for children comprising 3 mM epinephrine, 2.5 mM sodium metabisulfite, 3 mM tartrate, 9 mg/mL NaCl, 0.13 mg/mL EDTA or 0.16 mg/mL Na₂EDTA*2H₂O and pH 3.9.

Another particularly embodiment of the present invention is pharmaceutical composition used as junior formulation for children comprising 1 mg/mL epinephrine tartrate or 0.55 mg/mL epinephrine base and 0.45 mg/mL tartrate, 0.48 mg/mL sodium metabisulfite, 9 mg/mL NaCl, 0.13 mg/mL EDTA or 0.16 mg/mL Na₂EDTA*2H₂O and pH 3.9.

The pharmaceutical compositions of the present invention are adjusted to maintain iso-osmotic formulations by tonicity regulating agents such as glucose, glycerin, hydroxypropyl methyl cellulose, mannitol, polysorbate, propylene glycol, sodium bromide, sodium chloride, sodium iodide, sorbitol, urea, xylitol, and/or combinations thereof, preferably NaCl.

The pH of the pharmaceutical compositions of the present invention is adjusted by pH raising agents such as the acids or salt forms of one or more of lactate, tartrate, glutamate, malate, citrate, gluconate, benzoate, succinate, acetate, glycine, and aspartate, as well as lithium hydroxide, sodium hydroxide, potassium hydroxide, rubidium hydroxide, cesium hydroxide, calcium hydroxide, strontium hydroxide, and barium hydroxide, preferably sodium hydroxide and by pH lowering agents such as acetic acid, adipic acid, ascorbic acid, citric acid, hydrochloric acid, lactic acid, malic acid, monopotassium phosphate, monosodium phosphate, phosphoric acid, pyrophosphoric acid, succinic acid, sulfuric acid, and or tartaric acid, preferably hydrochloric acid.

Thus, a further embodiment of the present invention is a process for the preparation of a pharmaceutical composition according to the present invention, characterized in that epinephrine an antioxidant selected from the group consisting of sodium metabisulfite, sodium sulfite and sodium bisulfite, tartrate, NaCl and EDTA or Na₂EDTA*2H₂O together with water are brought into a suitable dosage form.

The present invention further concerns devices for delivery of the compositions, routes of administration, and methods for treating any medical condition for which epinephrine is suitable for alleviating at least one symptom. Although the epinephrine formulations of the present invention may be employed for any medical condition that would be improved thereby, in particular cases the medical condition for which the inventive composition is employed is an allergic reaction, including in the context of an allergic emergency, such as anaphylaxis, for example. Treatment of anaphylaxis concerns ameliorating or alleviating at least one symptom of anaphylaxis. In particular cases, the epinephrine formulation of the invention is employed to facilitate peripheral vascular resistance via alpha-stimulated vasoconstriction in cardiac dysrhythmias, such as cardiac arrest, that leads to impaired or totally inhibited cardiac output, such that blood is directed to the core of the body. Such a formulation and amount thereof is employed as long as there is no increased cardiac irritability to a medically unacceptable level.

Therefore, a further embodiment of the present invention is a pharmaceutical composition according to the present invention, for use in the treatment of physiological and/or pathophysiological states, selected from the group consisting of allergic reactions in the context of allergic emergencies and anaphylaxis and anaphylactoid reactions in the context of systemic toxic responses.

Accordingly, a further embodiment of the present invention is the use of a pharmaceutical composition according to the present invention for the preparation of a medicament for the treatment of physiological and/or pathophysiological states, selected from the group consisting of allergic reactions in the context of allergic emergencies and anaphylaxis and anaphylactoid reactions in the context of systemic toxic responses.

Another embodiment of the present invention is a method for the treatment of physiological and/or pathophysiological states, selected from the group consisting of allergic reactions in the context of allergic emergencies and anaphylaxis and anaphylactoid reactions in the context of systemic toxic responses by administering a medicament comprising a pharmaceutical composition according to the present invention.

Furthermore, the invention provides kits for treating epinephrine-required medical conditions, including allergic emergencies, such as anaphylaxis. The pharmaceutical compositions of the present invention provide surprisingly-enhanced stability over other formulations. The stability enhancements provide benefits at least in terms of patient safety, enhanced shelf-life, reduced waste, reduced cost, and/or improved convenience for the user. The compositions of the present invention provide formulations that are stable at room temperature and can be stored without the need for refrigeration. As such, the devices or kits can be placed on emergency crash carts and medical kits in clinics, emergency rooms, airplanes, schools, public places, restaurants, residences, on a person, or in urgent care centers or hospitals for easy access in emergency situations, for example.

Such treatment may be, and in most cases is, temporary, in particular embodiments of the invention. The formulations, methods, and kits of the invention are suitable for any setting in which epinephrine is required for medical purpose. In specific embodiments of the invention, the method or kit of the invention provides emergency relief from at least one symptom of anaphylaxis for a time sufficient for the patient to seek professional medical assistance. Thus, devices and kits of the invention are well-suited for inclusion in first aid kits in professional child care settings and homes, for example, especially where one or more persons at risk for anaphylaxis are known to dwell. They may also be conveniently carried by those who are at risk for anaphylaxis or those who are charged with caring for those who are at risk for anaphylaxis. They are also well-suited for inclusion in so called crash carts in medical emergency rooms. The methods of the invention are suitable for treating persons who are at risk for allergic emergencies, such as anaphylaxis, in any of the exemplary afore-mentioned settings.

Epinephrine is typically administered in anaphylaxis by injection under the skin, or into a muscle, although any route of administration may be suitable. Injections can be given by a health care professional in a clinic or hospital setting. Alternatively, an auto-injector form, for example, provides a convenient applicator for the health-care professional or for self-administration by patients who suffer a severe allergic response to certain stimuli.

Epinephrine is commonly administered parenterally by means of an injection device. Common injection devices range from a simple manual syringe system to an auto-injector. A manual syringe system would include a syringe comprising a barrel and a plunger and an appropriately-sized needle. Such simple syringes may be adapted to accept pre-filled cartridges, be packaged with the drug formulation loaded in the syringe, or used with vials, for example. Formulated drugs such as epinephrine may be prepared and filled into glass container, ampoules, prefilled cartridges, syringes, or vials that may be single or multi-dose containers, for example.

Thus, a further embodiment of the present invention is a glass syringe comprising a pharmaceutical composition according to the present invention. Another embodiment of the present invention is an auto-injector comprising a pharmaceutical composition according to the present invention. Another embodiment of the present invention is a set (kit) consisting of separate packs of
a) a glass syringe or an auto-injector and
b) a pharmaceutical composition according to the present invention, stored in a glass container, ampoule, prefilled cartridge or vials.

An exemplary epinephrine formulation for use in the treatment of the medical condition may be delivered by intramuscular injection, in specific embodiments. In specific embodiments, the injection device would provide 1.1 mL of the epinephrine formulation of the invention and deliver a single dose of 0.3 mL epinephrine from a 1:1000 dilution (0.3 mg) of a sterile solution (treatment of adults). Alternately, the injection device may provide 1.1 mL of the epinephrine formulation of the invention and deliver a single dose of 0.3 mL of epinephrine from a 1:2000 dilution (0.15 mg) of a sterile solution (treatment of children).

Automatic injectors or auto-injectors, such as those exemplary devices disclosed in U.S. Pat. Nos. 5,358,489; 5,400,644; 5,665,071; 5,695,472 and 9,186,459 for example, are known in the art. In general, all automatic injectors comprise a volume of epinephrine solution to be injected. In general, automatic injectors include a reservoir for holding the epinephrine solution, which is in fluid communication with a needle for delivering the drug, as well as a mechanism for automatically deploying the needle, inserting the needle into the patient, and delivering the dose into the patient. An illustrative and exemplary automatic injector is described in US 2005/0222539.

Exemplary injectors provide about 0.3 mL of epinephrine solution at about a concentration of either 0.5 or 1 mg of epinephrine per mL of solution (1:2000 or 1:1000, respectively). Each injector is capable of delivering a dose of epinephrine and any epinephrine left in the auto-injector (generally about 80% of the original volume of epinephrine) is unavailable for delivery and must be discarded.

Additionally, the auto-injectors deliver a uniform volume of 0.3 mL of epinephrine to the patient, whether that patient is an adult or a child. Whereas, the adult version delivers 0.3 mL of a 1:1000 dilution of epinephrine, the pediatric version delivers 0.3 mL of a 1:2000 dilution of epinephrine. This volume of medicine may present discomfort to smaller children, but any discomfort is offset by the life-saving nature of epinephrine in treating severe anaphylaxis. However, a further object of the invention is to fill the need for a composition and method of treating anaphylaxis in a person of less than about 30 kg, wherein a smaller dose of epinephrine can be delivered to the patient.

Thus, treatment of a medical condition, such as an allergic emergency that includes treatment of anaphylaxis, for which the invention is especially well-suited. In addition, treatment of allergic emergency includes treatment of other allergic conditions that may be treated with epinephrine. For example, the symptoms of anaphylactoid reactions to drugs closely mimic those of anaphylaxis and are treated in a similar manner. In cases where it is not clear whether the reaction is a systemic immunological response (anaphylaxis) or a systemic toxic response (anaphylactoid reaction), the accepted first line of treatment is with epinephrine. In this sense, treatment of an allergic emergency encompasses treatment of anaphylaxis, an anaphylactoid response or both.

In some embodiments, the present invention provides a method of treating an allergic emergency, such as anaphylaxis, in a patient. The method includes automatically injecting into a patient in need thereof a dose of 0.3 mg epinephrine consisting essentially of about 0.3 mL of an epinephrine solution. The concentration of epinephrine in the epinephrine solution is about 1 mg of epinephrine per mL of solution.

The smaller dose of epinephrine (0.15 mg dose in 0.3 mL), is especially suitable for treating smaller patients with body weights less than 30 kg. Thus, in some embodiments in which the dose is about 0.15 mg, the weight of the patient weighs less than about 30 kg. In particular embodiments, the patient weighs less than about 15 kg.

Pharmaceutical compositions of the present invention comprise an effective amount of one or more epinephrine formulations. The formulation may be dissolved or dispersed in a pharmaceutically acceptable carrier. The carrier may or may not be the stability-enhancing agent of the invention. The phrases “pharmaceutical or pharmacologically acceptable” refers to molecular entities and compositions that do not produce an adverse, allergic or other untoward reaction when administered to an animal, such as, for example, a human, as appropriate. The preparation of a pharmaceutical composition that contains at least one epinephrine formulation and/or additional active ingredient will be known to those of skill in the art considering the present disclosure, as exemplified by Remingon's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the pharmaceutical compositions is contemplated.

The epinephrine formulation may be administered in liquid form, and whether it need to be sterile for such routes of administration as injection. The present invention can be administered in any suitable manner although in specific embodiments its administration is intravenously, intradermally, intrathecally, intraarterially, intraperitoneally, intramuscularly, subcutaneously, locally, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

Upon formulation, solutions will be administered in a manner compatible with the dosage formulation and in such amount as is therapeutically effective. The formulations are easily administered in a variety of dosage forms such as formulated for parenteral administrations, such as injectable solutions. Further in accordance with the present invention, the composition of the present invention suitable for administration is provided in a pharmaceutically acceptable carrier with or without an inert diluent. The carrier should be assimilable and includes a liquid carrier. Except insofar as any conventional media, agent, diluent or carrier is detrimental to the recipient or to the therapeutic effectiveness of a composition contained therein, its use in administrable composition for use in practicing the methods of the present invention is appropriate. Examples of carriers or diluents include fats, oils, water, saline solutions, lipids, liposomes, resins, binders, fillers and the like, or combinations thereof. The composition may also comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

In accordance with the present invention, the composition is combined with the carrier in any convenient and practical manner, i.e., by solution, suspension, emulsification, admixture, encapsulation, absorption and the like. Such procedures are routine for those skilled in the art.

The pharmaceutical composition according to the invention can be used as medicaments in human or veterinary medicine. The patient or host can belong to any mammal species, for example a primate species, particularly humans; rodents, including mice, rats and hamsters; rabbits; horses, cattle, dogs, cats, etc. Animal models are of interest for experimental investigations, where they provide a model for the treatment of a human disease.

The actual dosage amount of a composition of the present invention administered to an animal patient can be determined by physical and physiological factors such as body weight, severity of condition, the type of disease being treated, previous or concurrent therapeutic interventions, idiopathy of the patient and on the route of administration. Depending upon the dosage and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject.

In certain embodiments, pharmaceutical compositions may comprise, for example, at least about 0.05% of an active compound. In other embodiments, the active compound may comprise between about 1% to about 75% of the weight of the unit, or between about 25% to about 60%, for example, and any range derivable therein. Naturally, the amount of active compound(s) in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In other non-limiting examples, a dose may also comprise from about 1 microgram/kg/body weight, about 5 microgram/kg body weight, about 10 microgram/kg/body weight, about 50 microgram/kg body weight, about 100 microgram/kg/body weight, about 200 microgram/kg body weight, about 350 microgram/kg/body weight, about 500 microgram/kg body weight, about 1 milligram/kg/body weight, about 5 milligram/kg body weight, about 10 milligram/kg/body weight, about 50 milligram/kg body weight, about 100 milligram/kg/body weight, about 200 milligram/kg body weight, about 350 milligram/kg/body weight, about 500 milligram/kg body weight, to about 1000 mg/kg/body weight or more per administration, and any range derivable therein. In non-limiting examples of a derivable range from the numbers listed herein, a range of about 5 mg/kg/body weight to about 100 mg/kg body weight, about 5 microgram/kg/body weight to about 500 milligram/kg body weight, etc., can be administered, based on the numbers described above.

In further embodiments, epinephrine formulations may be administered via a parenteral route. As used herein, the term “parenteral” includes routes that bypass the alimentary tract. Specifically, the pharmaceutical compositions disclosed herein may be administered for example, but not limited to intravenously, intradermally, intramuscularly, intraarterially, intrathecally, subcutaneously, or intraperitoneally.

Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy injectability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g. glycerol, propylene glycol, and liquid polyethylene glycol, and the like), suitable mixtures thereof, and/or vegetable oils. Proper fluidity may be maintained, for example, by using a coating, such as lecithin, by the maintenance of the required particle size in the case of dispersion and by using surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum mono stearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art considering the present disclosure. For example, one dosage may be dissolved in 1 mL of isotonic NaCl solution and either added to 1000 mL of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Sterile injectable solutions are prepared by incorporating the active compounds in the required amount in the appropriate solvent with various of the other ingredients enumerated above, as required, followed by sterile filtration. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. A powdered composition is combined with a liquid carrier such as, e. g. water or a saline solution, with or without a stabilizing agent.

Any of the compositions described herein may be comprised in a kit. In a non-limiting example, an epinephrine formulation of the invention may be comprised in a kit. The kits will thus comprise, a suitable container means and an epinephrine formulation of the present invention.

The kits may comprise a suitably aliquoted epinephrine formulation. In certain cases, the formulation comprises EDTA and one or more of acetylcysteine, cysteine, thioglycerol, or citric acid. The components of the kits may be packaged either in aqueous media or in lyophilized form. The container means of the kits will generally include at least one vial, test tube, flask, bottle, syringe or other container means, into which a component may be placed, and preferably, suitably aliquoted.

Where there are more than one component in the kit, the kit also will generally contain a second, third or other additional container into which the additional components may be separately placed. However, various combinations of components may be comprised in a vial. The kits of the present invention also will typically include a means for containing the epinephrine formulation and any other reagent containers in close confinement for commercial sale. Such containers may include injection or blow molded plastic containers into which the desired vials are retained.

When the components of the kit are provided in one and/or more liquid solutions, the liquid solution is an aqueous solution, with a sterile aqueous solution being particularly preferred. The epinephrine formulation may also be formulated into a syringeable composition. In which case, the container means may itself be a syringe, pipette, and/or other such like apparatus, from which the formulation may be applied to an appropriate area of the body, injected into an animal, and/or even applied to and/or mixed with the other components of the kit.

However, the components of the kit may be provided as dried powder(s). When reagents and/or components are provided as a dry powder, the powder can be reconstituted by the addition of a suitable solvent. It is envisioned that the solvent may also be provided in another container means.

Irrespective of the number and/or type of containers, the kits of the invention may also comprise, and/or be packaged with, an instrument for assisting with the injection/administration and/or placement of the ultimate epinephrine formulation into the body of an animal. Such an instrument may be a syringe, auto-injector, or any such medically approved injection delivery vehicle.

Epinephrine can be used in its final non-salt form. On the other hand, the present invention also encompasses the use of epinephrine in the form of its pharmaceutically acceptable salts, which can be derived from various organic and inorganic bases by procedures known in the art. Suitable acid-addition salts are inorganic or organic salts of all physiologically or pharmacologically acceptable acids, for example halides, in particular hydrochlorides or hydrobromides, lactates, sulfates, citrates, tartrates, maleates, fumarates, oxalates, acetates, phosphates, methyl-sulfonates or p-toluenesulfonates. Epinephrine tartrate is the preferred pharmaceutically acceptable salt form. Pharmaceutically acceptable salt forms of epinephrine are for the most part prepared by conventional methods.

Even without further embodiments, it is assumed that a person skilled in the art will be able to use the above description in the broadest scope. The preferred embodiments should therefore merely be regarded as descriptive disclosure which is absolutely not limiting in any way.

The following examples are thus intended to explain the invention without limiting it. Unless indicated otherwise, percent data denote percent by weight. All temperatures are indicated in degrees Celsius.

### EXAMPLE 1: CHIRAL HPLC FOR DETERMINATION OF ENANTIOMERIC RATIO (D- VERSUS L-EPINEPHRINE)

Epinephrine solutions were either directly subjected to chiral HPLC (0.55 mg/mL epinephrine formulations) or diluted 1:2 by placebo solution (1.1 mg/mL epinephrine formulations). Chromatographic separation was conducted on a Vanquish Flex system (Thermo Scientific). 2 μl sample were applied on a chiral HPLC column (Orpak CDBS-453, 4.6 mm×150 mm, 3 μm particle size, Shodex). Chromatographic separation was conducted in Eluent A (99% [v/v] 10 mM ammonium acetate, pH 4.0; 1% [v/v] acetonitrile) at 15° C. over 35 min at a flow rate of 0.6 mL/min. UV-detection of L- and D-epinephrine was carried out at 280 nm. Corresponding peak areas were integrated and the enantiomeric ratio calculated.

### EXAMPLE 2: PGC (POROUS GRAPHITIC CARBON) HPLC FOR QUANTIFICATION OF TOTAL EPINEPHRINE AND ESA

Epinephrine solutions were either directly subjected to PGC-HPLC (0.55 mg/mL epinephrine formulations) or diluted 1:2 by placebo solution (1.1 mg/mL epinephrine formulations). Chromatographic separation was conducted on a Vanquish Flex system (Thermo Scientific) with flow rates of 0.5 to 1.5 mL/min. 2 μl sample were applied on a PGC column (HYPERCARB PGC, 4.6 mm×100 mm, 3 μm particle size, Thermo Scientific). Chromatographic separation was conducted over 60 min at 60° C. using a gradient from 0.2% TFA to 0.05% TFA 75% acetonitrile and then a gradient to 100% isopropanol and finally a gradient to 0.2% TFA. UV-detection of epinephrine and ESA was carried out at 280 nm. Corresponding peak areas were integrated and amounts of epinephrine and ESA calculated considering the respective UV response factors and using an external calibration curve.

### EXAMPLE 3: HPIC (HIGH PERFORMANCE ION CHROMATOGRAPHY) FOR DETERMINATION OF SODIUM METABISULFITE AS SULFITE

Epinephrine formulations were diluted 1:100 (formulations with 1.67 mg/mL Na₂S₂O₅), 1:50 (formulations with 0.95 mg/mL and 0.48 mg/mL Na₂S₂O₅) and 1:25 (formulation with 0.16 mg/mL Na₂S₂O₅) in water containing 0.37% formaldehyde. Chromatographic separation was conducted on a Dionex Ion chromatograph ICS3000. 50 μl sample were applied on a HPIC column (AS4aSC, 4 mm×250 mm Thermo Scientific) using a guard column (AG4aSC, 4 mm×50 mm Thermo Scientific). Chromatographic separation was conducted in Eluent A (1.3 mM sodium hydrogen carbonate and 1.4 mM sodium carbonate in water) at 30° C. over 20 min at a flow rate of 2 mL/min. Conductivity detection with suppressor technology was used at a cell temperature of 35° C. Corresponding peak areas were integrated and amounts of sulfite calculated using an external calibration curve.

### EXAMPLE 4: ALTERNATIVE QUANTIFICATION OF TOTAL EPINEPHRINE AND ESA (ONLY EXAMPLE 11, FIGS. 18-21)

### A: HPLC Quantification of Total Epinephrine

HPLC quantification of total epinephrine was conducted according to USP Epinephrine Injection using a chromatographic system equipped with a UV 280 nm detector and a 4.6 mm×15 cm column containing L7 packaging.

### B: ESA Quantification

Epinephrine solutions were either directly subjected to C18-HPLC (0.55 mg/mL epinephrine formulations) or diluted 1:2 by placebo solution (1.1 mg/mL epinephrine formulations). Chromatographic separation was conducted on a Agilent 1260 system with a flow rate of 0.8 mL/min. 50 μl sample were applied on a C18 column (Kromasil 100-5-C18, 4.6 mm×250 mm, 5 μm particle size, Sigma-Aldrich). Chromatographic separation was conducted over 57 min at 30° C. using a gradient from 0.01 M sodium-1-heptansulfonate monohydrate to 0.005 M sodium-1-heptansulfonate monohydrate 50% acetonitrile and then a gradient to 0.01 M sodium-1-heptansulfonate monohydrate. UV-detection of ESA was carried out at 280 nm. The corresponding peak area was integrated and the amounts of ESA calculated relative to the USP impurity F reference standard.

### EXAMPLE 5: ADDITION OF EDTA DECREASES FORMATION OF D-EPINEPHRINE AND ESA AND STABILIZES TOTAL AND L-EPINEPHRINE IN THE JUNIOR AND THE SENIOR FORMULATION OF EPIPEN

| | |
|---|---|
| Junior formulation | 0.55 mg/mL epinephrine base |
| | 6 mg/mL NaCl |
| | 1.67 mg/mL sodium metabisulfite |
| | +/−Na₂EDTA*2H₂O (0.16 mg/mL) |
| | hydrochloric acid as required for pH 3.4 |
| Senior formulation | 1.1 mg/mL epinephrine base |
| | 6 mg/mL NaCl |
| | 1.67 mg/mL sodium metabisulfite |
| | +/−Na₂EDTA*2H₂O (0.16 mg/mL) |
| | hydrochloric acid as required for pH 3.4 |

### Legend FIG. 1:

Commercially available epinephrine formulations (junior: 0.55 mg/mL and senior: 1.1 mg/mL) were incubated at 60° C. for 28 days in the presence or absence of 0.16 mg/mL Na₂EDTA*2H₂O. For each formulation, the enantiomer ratio was determined by chiral HPLC after 6, 14 and 28 days. The D-epinephrine content is given relative to the total amount of epinephrine in the samples. E:S=molar ratio of epinephrine to sulfite equivalents (SO₃²⁻).

### Legend FIG. 2:

Commercially available epinephrine formulations (junior: 0.55 mg/mL and senior: 1.1 mg/mL) were incubated at 60° C. for 28 days in the presence or absence of 0.16 mg/mL Na₂EDTA*2H₂O. For each formulation, the total ESA content was determined by HPLC after 6, 14 and 28 days. E:S=molar ratio of epinephrine to sulfite equivalents (SO₃²⁻).

### Results Shown in FIGS. 1 and 2:

By addition of the chelator EDTA to the formulation of Epipen (stored in a SyriQ syringe), the amount of impurities D-epinephrine & ESA formed is significantly reduced for both, the junior (0.55 mg/mL) and the senior (1.1 mg/mL) strength.

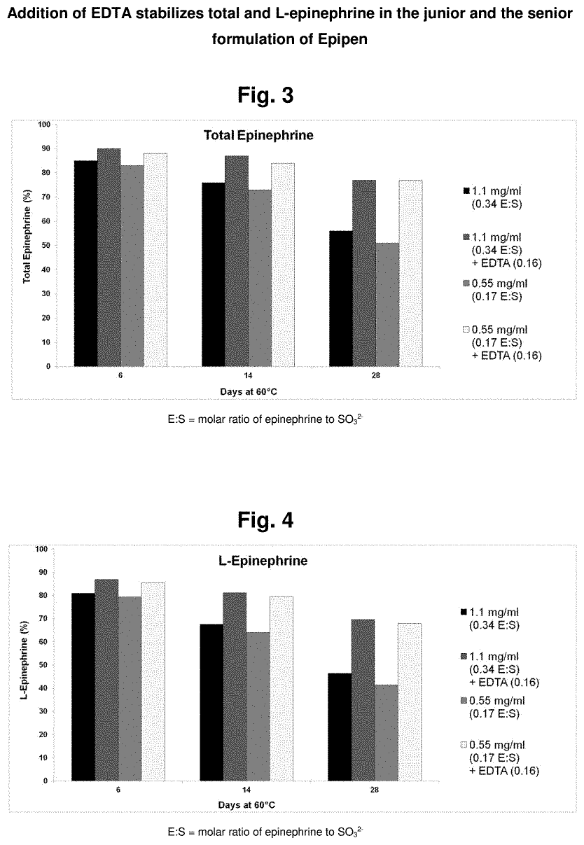

### Legend FIG. 3:

Commercially available epinephrine formulations (junior: 0.55 mg/mL and senior: 1.1 mg/mL) were incubated at 60° C. for 28 days in the presence or absence of 0.16 mg/mL Na₂EDTA*2H₂O. For each formulation, the total epinephrine content was determined after 6, 14 and 28 days by HPLC. Values are given relative to the starting amount of epinephrine (0.55 and 1.1. mg/mL, respectively). E:S=molar ratio of epinephrine to sulfite equivalents (SO₃²⁻).

### Legend FIG. 4:

Commercially available epinephrine formulations (junior: 0.55 mg/mL and senior: 1.1 mg/mL) were incubated at 60° C. for 28 days in the presence or absence of 0.16 mg/mL Na₂EDTA*2H₂O. Based on the quantification of total epinephrine and the determination of the enantiomer ratio, the L-epinephrine content of each sample was calculated. Values are given relative to the starting amount of epinephrine (0.55 and 1.1. mg/mL, respectively). E:S=molar ratio of epinephrine to sulfite equivalents (SO₃²⁻).

### Results Shown in FIGS. 3 and 4:

By addition of the chelator EDTA to the formulation of Epipen (stored in a SyriQ syringe), stability of total (D- & L-) and L-epinephrine is significantly increased for both, the junior (0.55 mg/mL) and the senior (1.1 mg/mL) strength.

### EXAMPLE 6: ADDITION OF CHELATORS TARTRATE & EDTA DECREASES FORMATION OF D-EPINEPHRINE LONG TERM, REDUCES ESA FORMATION AND STABILIZES TOTAL AND L-EPINEPHRINE

| | |
|---|---|
| Senior formulation | 1.1 mg/mL epinephrine base |
| | or 2 mg/mL epinephrine tartrate* |
| | 6 mg/mL NaCl |
| | 1.67 mg/mL sodium metabisulfite |
| | +/−Na₂EDTA*2H₂O (0.16 mg/mL) |
| | hydrochloric acid/NaOH as required |
| | for pH 3.9 |

*contains 1.1 mg/mL epinephrine base

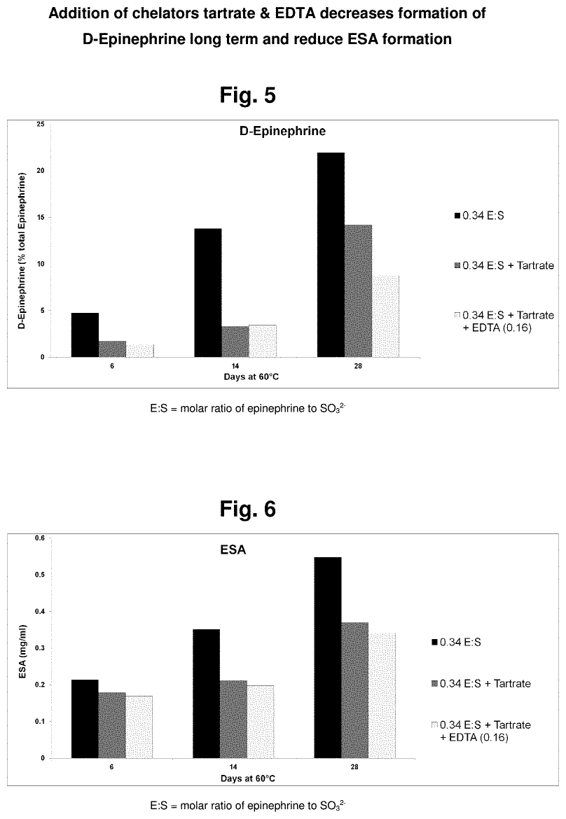

### Legend FIG. 5:

Different epinephrine senior formulations (all 1.1 mg/mL and E:S: 0.34) were incubated at 60° C. for 28 days in the presence or absence of 0.16 mg/mL Na₂EDTA*2H₂O and 0.9 mg/mL tartrate. For each formulation, the enantiomer ratio was determined by chiral HPLC after 6, 14 and 28 days. The D-epinephrine content is given relative to the total amount of epinephrine in the samples. E:S=molar ratio of epinephrine to sulfite equivalents (SO₃²⁻).

### Legend FIG. 6:

Different epinephrine senior formulations (all 1.1 mg/mL and E:S: 0.34) were incubated at 60° C. for 28 days in the presence or absence of 0.16 mg/mL Na₂EDTA*2H₂O and 0.9 mg/mL tartrate. For each formulation, the total ESA content was determined by HPLC after 6, 14 and 28 days. E:S=molar ratio of epinephrine to sulfite equivalents (SO₃²⁻).

### Results Shown in FIGS. 5 and 6:

Already by use of epinephrine tartrate (the chelator tartrate is used for pH stabilization) D-enantiomer formation is significantly reduced in 1.1 mg/mL epinephrine formulations. However, addition of tartrate alone is not sufficient for long term stability. Only by further addition of EDTA, long term stability is given. Furthermore, ESA formation is reduced by addition of tartrate and EDTA.

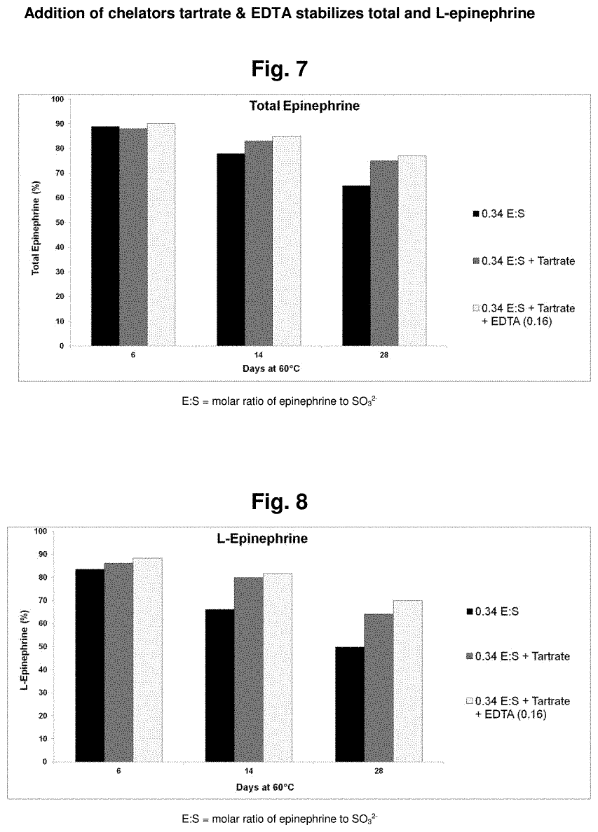

### Legend FIG. 7:

Different epinephrine senior formulations (all 1.1 mg/mL and E:S: 0.34) were incubated at 60° C. for 28 days in the presence or absence of 0.16 mg/mL Na₂EDTA*2H₂O and 0.9 mg/mL tartrate. For each formulation, the total epinephrine content was determined after 6, 14 and 28 days by HPLC. Values are given relative to the starting amount of epinephrine (1.1 mg/mL). E:S=molar ratio of epinephrine to sulfite equivalents (SO₃²⁻).

### Legend FIG. 8:

Different epinephrine senior formulations (all 1.1 mg/mL and E:S: 0.34) were incubated at 60° C. for 28 days in the presence or absence of 0.16 mg/mL Na₂EDTA*2H₂O and 0.9 mg/mL tartrate. For each formulation, the enantiomer ratio was determined by chiral HPLC after 6, 14 and 28 days. Based on the quantification of total epinephrine (D&L) and the enantiomer ratio, the L-epinephrine content of each sample was calculated. Values are given relative to the starting amount of epinephrine (1.1 mg/mL). E:S=molar ratio of epinephrine to sulfite equivalents (SO₃²⁻).

### Results Shown in FIGS. 7 and 8:

By use of epinephrine tartrate & EDTA stability of total (D- & L-) and L-epinephrine is significantly increased in 1.1 mg/mL epinephrine formulations.

### EXAMPLE 7: ADDITION OF CHELATORS TARTRATE & EDTA DECREASES FORMATION OF D-EPINEPHRINE AND ESA OVER A WIDE METABISULFITE CONCENTRATION RANGE

| | |
|---|---|
| Senior formulation | 1.1 mg/mL epinephrine base |
| | or 2 mg/mL epinephrine tartrate* |
| | 6-8.4 mg/mL NaCl** |
| | 0.2-1.67 mg/mL sodium metabisulfite (1-8.7 mM) |
| | +/−Na₂EDTA*2H₂O (0.05-0.4 mg/mL) |
| | hydrochloric acid/NaOH as required for pH 3.9 |

*contains 1.1 mg/mL epinephrine base
**NaCl concentrations were adjusted to maintain iso-osmotic formulations

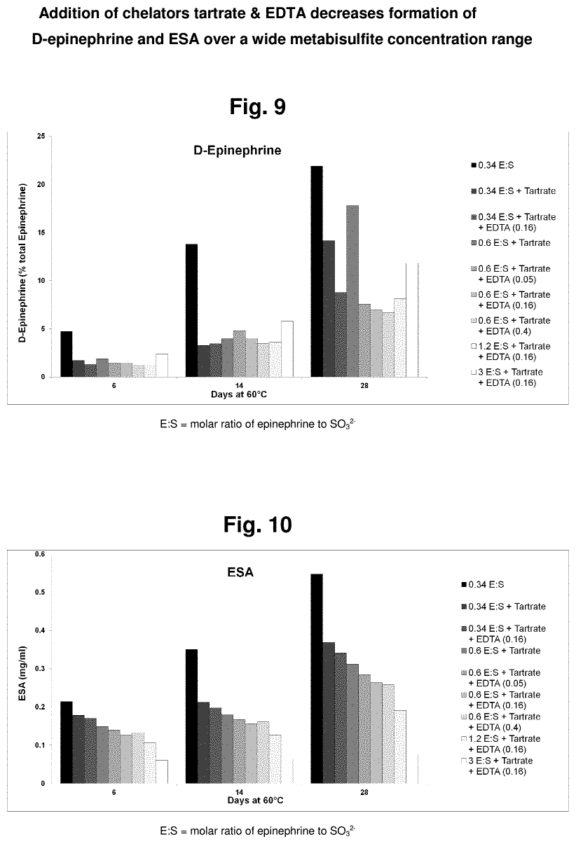

### Legend FIG. 9:

Different epinephrine senior formulations (all 1.1 mg/mL and E:S: 0.34 to 3.00) were incubated at 60° C. for 28 days in the presence or absence of Na₂EDTA*2H₂O (0.05-0.4 mg/mL) and 0.9 mg/mL tartrate. For each formulation, the enantiomer ratio was determined by chiral HPLC after 6, 14 and 28 days. The D-epinephrine content is given relative to the total amount of epinephrine in the samples. E:S=molar ratio of epinephrine to sulfite equivalents (SO₃²⁻).

### Legend FIG. 10:

Different epinephrine senior formulations (all 1.1 mg/mL and E:S: 0.34 to 3.00) were incubated at 60° C. for 28 days in the presence or absence of Na₂EDTA*2H₂O (0.05-0.4 mg/mL) and 0.9 mg/mL tartrate. For each formulation, the total ESA content was determined by HPLC after 6, 14 and 28 days. E:S=molar ratio of epinephrine to sulfite equivalents (SO₃²⁻).

### Results Shown in FIGS. 9 and 10:

Epinephrine formulations (1.1 mg/mL) with different E:S ratios show in the presence of tartrate and EDTA reduced D-epinephrine levels. For long term L-epinephrine stability, addition of tartrate alone is not sufficient. The optimal E:S ratio to prevent racemization is 0.6.

ESA formation is highly dependent on the E:S ratio. The lower the ratio, the less ESA is formed. However, already an E:S ratio of around 1.2 can result in oxidative damage due to too low antioxidant amounts (see Emerade® recall [described above] and sodium metabisulfite quantification [shown below]).

### EXAMPLE 8: ADDITION OF CHELATORS TARTRATE & EDTA TOGETHER WITH A HIGH E:S RATIO STABILIZES TOTAL AND L-EPINEPHRINE

| | |
|---|---|
| Senior formulation | 1.1 mg/mL epinephrine base |
| | or 2 mg/mL epinephrine tartrate* |
| | 6-8.4 mg/mL NaCl** |
| | 0.2-1.67 mg/mL sodium metabisulfite (1-8.7 mM) |
| | +/−Na₂EDTA*2H₂O (0.05-0.4 mg/mL) |
| | hydrochloric acid/NaOH as required for pH 3.9 |

*contains 1.1 mg/mL epinephrine base
**NaCl concentrations were adjusted to maintain iso-osmotic formulations

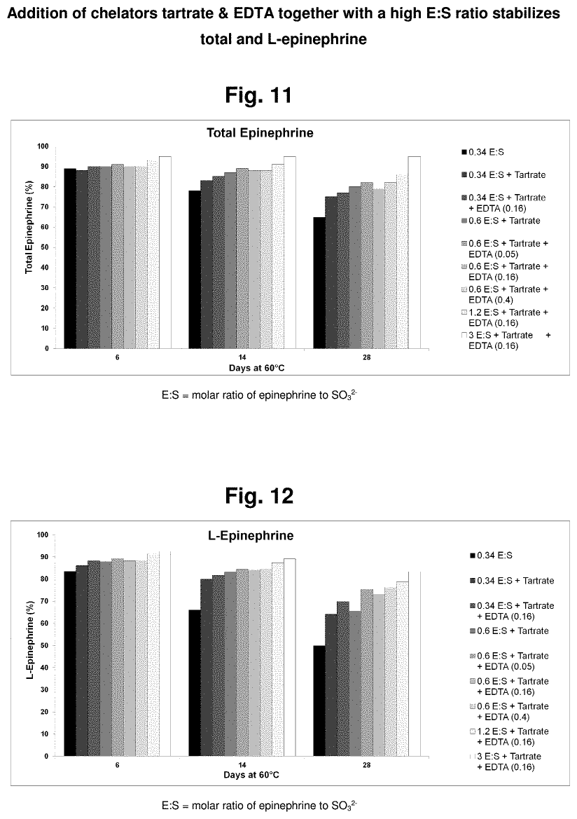

### Legend FIG. 11:

Different epinephrine senior formulations (all 1.1 mg/mL and E:S: 0.34 to 3.00) were incubated at 60° C. for 28 days in the presence or absence of Na₂EDTA*2H₂O (0.05-0.4 mg/mL) and 0.9 mg/mL tartrate. For each formulation, the total epinephrine content was determined after 6, 14 and 28 days by HPLC. Values are given relative to the starting amount of epinephrine (1.1 mg/mL). E:S=molar ratio of epinephrine to sulfite equivalents (SO₃²⁻).

### Legend FIG. 12:

Different epinephrine senior formulations (all 1.1 mg/mL and E:S: 0.34 to 3.00) were incubated at 60° C. for 28 days in the presence or absence of Na₂EDTA*2H₂O (0.05-0.4 mg/mL) and 0.9 mg/mL tartrate. For each formulation, the enantiomer ratio was determined by chiral HPLC after 6, 14 and 28 days. Based on the quantification of total epinephrine (D&L) and the enantiomer ratio, the L-epinephrine content of each sample was calculated. Values are given relative to the starting amount of epinephrine (1.1 mg/mL). E:S=molar ratio of epinephrine to sulfite equivalents (SO₃²⁻).

### Results Shown in FIGS. 11 and 12:

Epinephrine formulations (1.1 mg/mL) with different E:S ratios are stabilized by addition of tartrate and EDTA regarding total and L-epinephrine. Furthermore, total and L-epinephrine stability increases with increasing E:S ratio.

However, already an E:S ratio of around 1.2 can result in oxidative damage due to too low antioxidant amounts (see Emerade® recall [described above] and sodium metabisulfite quantification [shown below]).

Thus, formulations with an E:S ratio=0.6 are regarded optimal for long term stability.

### EXAMPLE 9: THE CONCENTRATION OF THE ANTIOXIDANT SODIUM METABISULFITE DECREASES DRASTICALLY OVER TIME

| | |
|---|---|
| Senior formulation | 1.1 mg/mL epinephrine base |
| | or 2 mg/mL epinephrine tartrate* |
| | 6-8.4 mg/mL NaCl** |
| | 0.2-1.67 mg/mL sodium metabisulfite (1-8.7 mM) |
| | +/−Na₂EDTA*2H₂O (0.05-0.4 mg/mL) |
| | hydrochloric acid/NaOH as required for pH 3.9 |

*contains 1.1 mg/mL epinephrine base
**NaCl concentrations were adjusted to maintain iso-osmotic formulations

Legend FIG. 13:

Different epinephrine senior formulations (all 1.1 mg/mL and E:S: 0.34 to 3.00) were incubated at 60° C. for 28 days in the presence or absence of EDTA (0.05-0.4 mg/mL) and 0.9 mg/mL tartrate. For each formulation, the sulfite amount was determined by HPIC after 28 days and the residual sodium metabisulfite concentration calculated. E:S=molar ratio of epinephrine to sulfite equivalents (SO₃²⁻).

### Results Shown in FIG. 13:

In all tested formulations, the antioxidant concentration decreases drastically over time. However, at E:S ratios 1.2 residual sodium metabisulfite levels are so low that oxidative damage is likely to occur towards the end of shelf-life.

Thus, formulations with an E:S ratio=0.6 are regarded optimal for long term stability.

### EXAMPLE 10: ADDITION OF TARTRATE & EDTA TOGETHER WITH AN E:S RATIO OF 0.6 DECREASE FORMATION OF D-EPINEPHRINE AND ESA AND STABILIZES BOTH, TOTAL AND L-EPINEPHRINE ALSO IN JUNIOR FORMULATIONS

| | |
|---|---|
| Junior formulation | 0.55 mg/mL epinephrine base |
| | or 1 mg/mL epinephrine tartrate* |
| | 6-9 mg/mL NaCl** |
| | 0.48-1.67 mg/mL sodium |
| | metabisulfite (2.5-8.7 mM) |
| | +/−Na₂EDTA*2H₂O (0.05-0.4 |
| | mg/mL) |
| | hydrochloric acid/NaOH as required |
| | for pH 3.9 |

*contains 0.55 mg/mL epinephrine base
**NaCl concentrations were adjusted to maintain iso-osmotic formulations

### Legend FIG. 14:

Different epinephrine junior formulations (all 0.55 mg/mL and E:S: 0.17 to 0.6) were incubated at 60° C. for 28 days in the presence or absence of Na₂EDTA*2H₂O (0.05-0.4 mg/mL) and 0.45 mg/mL tartrate. For each formulation, the enantiomer ratio was determined by chiral HPLC after 6, 14 and 28 days. The D-epinephrine content is given relative to the total amount of epinephrine in the samples. E:S=molar ratio of epinephrine to sulfite equivalents (SO₃²⁻).

### Legend FIG. 15:

Different epinephrine junior formulations (all 0.55 mg/mL and E:S: 0.17 to 0.6) were incubated at 60° C. for 28 days in the presence or absence of Na₂EDTA*2H₂O (0.05-0.4 mg/mL) and 0.45 mg/mL tartrate. For each formulation, the total ESA content was determined by HPLC after 6, 14 and 28 days. E:S=molar ratio of epinephrine to sulfite equivalents (SO₃²⁻).

### Results Shown in FIGS. 14 and 15:

Also in 0.55 mg/mL epinephrine formulations (junior strength) D-Epinephrine is reduced by addition of tartrate and EDTA and adjustment of E:S ratio to 0.6.

For long term L-epinephrine stability, addition of tartrate alone is insufficient.

In contrast, for reduction of ESA formation, addition of tartrate is already sufficient.

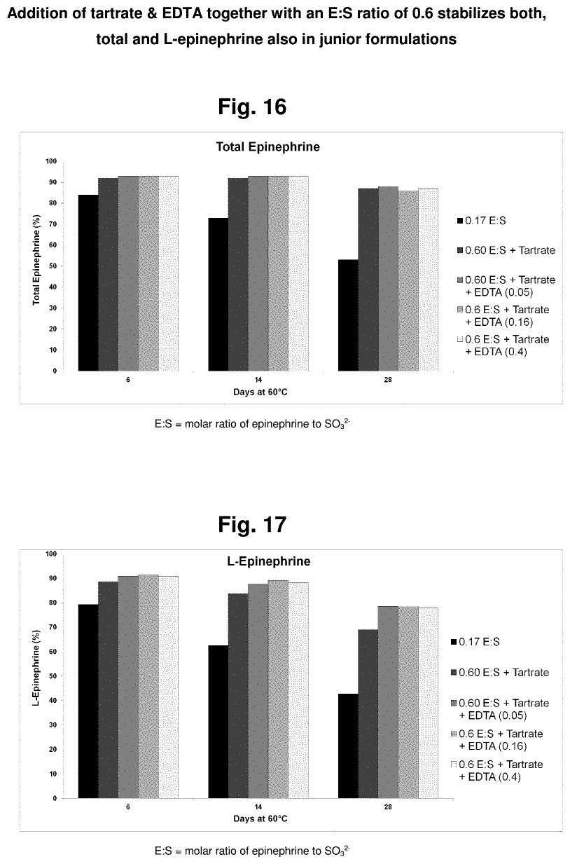

### Legend FIG. 16:

Different epinephrine junior formulations (all 0.55 mg/mL and E:S: 0.17 to 0.6) were incubated at 60° C. for 28 days in the presence or absence of Na₂EDTA*2H₂O (0.05-0.4 mg/mL) and 0.45 mg/mL tartrate. For each formulation, the total epinephrine content was determined after 6, 14 and 28 days by HPLC. Values are given relative to the starting amount of epinephrine (0.55 mg/mL). E:S=molar ratio of epinephrine to sulfite equivalents (SO₃²⁻).

### Legend FIG. 17:

Different epinephrine junior formulations (all 0.55 mg/mL and E:S: 0.17 to 0.6) were incubated at 60° C. for 28 days in the presence or absence of Na₂EDTA*2H₂O (0.05-0.4 mg/mL) and 0.45 mg/mL tartrate. For each formulation, the enantiomer ratio was determined by chiral HPLC after 6, 14 and 28 days. Based on the quantification of total epinephrine (D&L) and the enantiomer ratio, the L-epinephrine content of each sample was calculated. Values are given relative to the starting amount of epinephrine (0.55 mg/ml). E:S=molar ratio of epinephrine to sulfite equivalents (SO₃²⁻).

### Results Shown in FIGS. 16 and 17:

Total Epinephrine in 0.55 mg/mL epinephrine formulation is stabilized by addition of Tartrate and EDTA and increase of E:S ratio. At an E:S ratio of 0.6 high stability is given.

For stabilization of L-Epinephrine the presence of both, tartrate and EDTA together with a high E:S ratio is essential.

### EXAMPLE 11: AN E:S RATIO OF 0.6 AND ADDITION OF CHELATORS TARTRATE & EDTA DECREASES FORMATION OF D-EPINEPHRINE AND ESA AND STABILIZES BOTH, TOTAL AND L-EPINEPHRINE ALSO UPON LONG TERM STORAGE AT ROOM TEMPERATURE

| | |
|---|---|
| Senior | 1.1 mg/mL epinephrine base |
| formulation | or 2 mg/mL epinephrine tartrate* |
| | 6 mg/mL NaCl |
| | 0.95-1.67 mg/mL sodium metabisulfite (5-8.7 mM) |
| | +/−Na₂EDTA*2H₂O (0.16 mg/mL) |
| | hydrochloric acid/NaOH as required for pH 3.9 |
| Junior | 0.55 mg/mL epinephrine base |
| formulation | or 1 mg/mL epinephrine tartrate* |
| | 6 mg/mL NaCl |
| | 0.48-1.67 mg/mL sodium metabisulfite (2.5-8.7 mM) |
| | +/−Na₂EDTA*2H₂O (0.16 mg/mL) |
| | hydrochloric acid/NaOH as required for pH 3.9 |

*contains 1.1 mg/mL (senior) 0.55 mg/mL (junior) epinephrine base

### Legend FIG. 18:

Two epinephrine formulations (1.1 mg/mL [E:S: 0.34] and 0.55 mg/mL [E:S: 0.17] were incubated at 25° C. for 12 months together with two optimized epinephrine formulations (1.1 mg/mL and 0.55 mg/mL [both E:S: 0.6]), which contain Na₂EDTA*2H₂O (0.16 mg/mL) and tartrate (0.45 mg/mL). For each formulation the enantiomer ratio was determined by chiral HPLC after 6 and 12 months. The D-epinephrine content is given relative to the total amount of epinephrine in the samples.

E:S=molar ratio of epinephrine to sulfite equivalents (SO₃²⁻).

### Legend FIG. 19:

Two epinephrine formulations (1.1 mg/mL [E:S: 0.34] and 0.55 mg/mL [E:S: 0.17] were incubated at 25° C. for 12 months together with two optimized epinephrine formulations (1.1 mg/mL and 0.55 mg/mL [both E:S: 0.6]), which contain Na₂EDTA*2H₂O (0.16 mg/mL) and tartrate (0.45 mg/mL). For each formulation the total ESA content was determined by HPLC after 6 and 12 months.

E:S=molar ratio of epinephrine to sulfite equivalents (SO₃²⁻).

### Results Shown in FIGS. 18 and 19:

Upon storage at room temperature for 12 months epinephrine formulations with an E:S ratios of 0.6 (0.55 mg/mL and 1.1 mg/mL) containing tartrate and EDTA show reduced D-epinephrine levels compared to formulations with lower E:S ratio and without chelators. Also, ESA formation is reduced in the optimized formulations. Thus, room temperature data confirm the superiority of the optimized formulations regarding racemization and ESA formation.

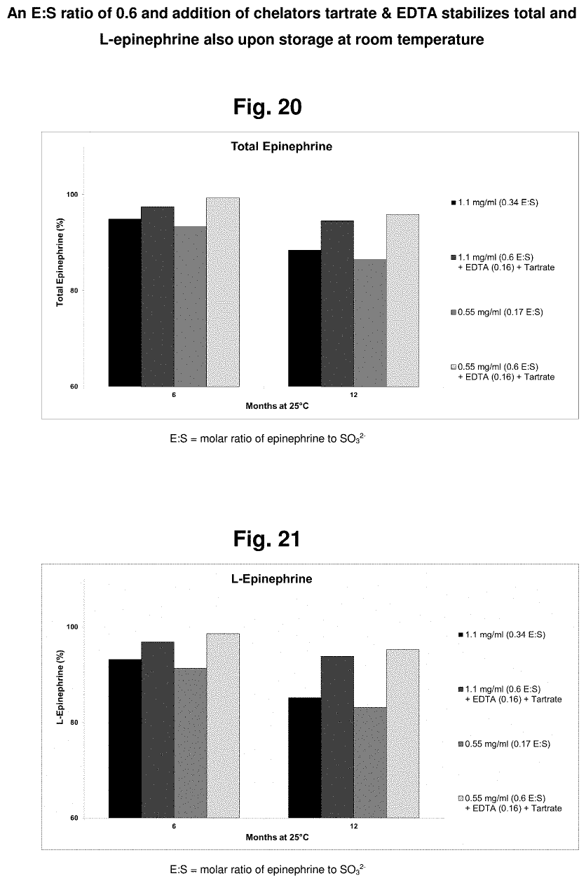

### Legend FIG. 20:

Two epinephrine formulations (1.1 mg/mL [E:S: 0.34] and 0.55 mg/mL [E:S: 0.17] were incubated at 25° C. for 12 months together with two optimized epinephrine formulations (1.1 mg/mL and 0.55 mg/mL [both E:S: 0.6]), which contain Na₂EDTA*2H₂O (0.16 mg/mL) and tartrate (0.45 mg/mL). For each formulation the total epinephrine content was determined after 6 and 12 months by HPLC. Values are given relative to the starting amount of epinephrine (1.1 mg/ml or 0.55 mg/mL, respectively).

E:S=molar ratio of epinephrine to sulfit equivalents (SO₃²⁻).

### Legend FIG. 21:

Two epinephrine formulations (1.1 mg/mL [E:S: 0.34] and 0.55 mg/mL [E:S: 0.17] were incubated at 25° C. for 12 months together with two optimized epinephrine formulations (1.1 mg/mL and 0.55 mg/mL [both E:S: 0.6]), which contain Na₂EDTA*2H₂O (0.16 mg/mL) and tartrate (0.45 mg/mL). For each formulation the enantiomer ratio was determined by chiral HPLC after 6 and 12 months. Based on the quantification of total epinephrine (D&L) and the enantiomer ratio, the L-epinephrine content of each sample was calculated. Values are given relative to the starting amount of epinephrine (1.1 mg/ml or 0.55 mg/mL, respectively).

E:S=molar ratio of epinephrine to sulfite equivalents (SO₃²⁻).

### Results Shown in FIGS. 20 and 21:

Upon storage at room temperature for 12 months epinephrine formulations with an E:S ratios of 0.6 (0.55 mg/mL and 1.1 mg/mL) containing tartrate and EDTA show increased total epinephrine levels compared to formulations with lower E:S ratio and without chelators. L-epinephrine levels are significantly higher in the optimized formulations than in the competitor. Thus, room temperature data confirm the superiority of the optimized formulations.

1. Pharmaceutical composition comprising epinephrine and an antioxidant selected from the group consisting of sodium metabisulfite, sodium sulfite and sodium bisulfite in a molar ratio of epinephrine to sulfite equivalents of 0.9-0.1, 6-10 mg/mL of a tonicity regulating agent, 0.04-0.31 mg/mL EDTA or 0.05-0.4 mg/mL Na₂EDTA*2H₂O and pH 3-4.5, wherein the concentration of epinephrine is 2-8 mM.

2. Pharmaceutical composition according to claim 1, compositions comprising epinephrine and an antioxidant selected from the group consisting of sodium metabisulfite, sodium sulfite and sodium bisulfite in a molar ratio of epinephrine to sulfite equivalents of 0.8-0.15, tartrate in a molar ratio of epinephrine to tartrate of 0.8-1.2, 6-10 mg/mL of a tonicity regulating agent, 0.04-0.31 mg/mL EDTA or 0.05-0.4 mg/mL Na₂EDTA*2H₂O and pH 3-4.5, wherein the concentration of epinephrine is 2-4 or 4-8 mM.

3. Pharmaceutical composition according to claim 1, comprising epinephrine and an antioxidant selected from the group consisting of sodium metabisulfite, sodium sulfite and sodium bisulfite in a molar ratio of epinephrine to sulfite equivalents of 0.7-0.2, tartrate in a molar ratio of epinephrine to tartrate of 0.8-1.2, 8-9.5 mg/mL of a tonicity regulating agent, 0.08-0.24 mg/mL EDTA or 0.1-0.3 mg/mL Na₂EDTA*2H₂O and pH 3.3-4.2, wherein the concentration of epinephrine is 2-4 or 5-7 mM.

4. Pharmaceutical composition comprising 4-8 mM epinephrine, an antioxidant selected from the group consisting of 3-9 mM sodium metabisulfite, 6-18 mM sodium sulfite and 6-18 mM sodium bisulfite, 6-10 mg/mL of a tonicity regulating agent, 0.04-0.31 mg/mL EDTA or 0.05-0.4 mg/mL Na₂EDTA*2H₂O and pH 3-4.5.

5. Pharmaceutical composition according to claim 4, comprising 5-7 mM epinephrine, an antioxidant selected from the group consisting of 3.5-7 mM sodium metabisulfite, 7-14 mM sodium sulfite and 7-14 mM sodium bisulfite, 5-7 mM tartrate, 8-9 mg/mL of a tonicity regulating agent, 0.08-0.24 mg/mL EDTA or 0.1-0.3 mg/mL EDTA and pH 3.3-4.2.

6. Pharmaceutical composition according to claim 4, comprising 5.5-6.5 mM epinephrine, an antioxidant selected from the group consisting of 4.5-5.5 mM sodium metabisulfite, 9-11 mM sodium sulfite and 9-11 mM sodium bisulfite, 5.5-6.5 mM tartrate, 8-9 mg/mL NaCl, 0.09-0.16 mg/mL EDTA or 0.12-0.2 mg/mL Na₂EDTA*2H₂O and pH 3.3-4.2.

7. Pharmaceutical composition comprising 2-4 mM epinephrine, an antioxidant selected from the group consisting of 1.5-9 mM sodium metabisulfite, 3-18 mM sodium sulfite and 3-18 mM sodium bisulfite, 6-10 mg/mL of a tonicity regulating agent, 0.04-0.31 mg/mL EDTA or 0.05-0.4 mg/mL Na₂EDTA*2H₂O and pH 3-4.5.

8. Pharmaceutical composition according to claim 7, comprising 2-4 mM epinephrine, an antioxidant selected from the group consisting of 1.7-7 mM sodium metabisulfite, 3.4-14 mM sodium sulfite and 3.4-14 mM sodium bisulfite, 2-4 mM tartrate, 8.5-9.5 mg/mL of a tonicity regulating agent, 0.08-0.24 mg/mL EDTA or 0.1-0.3 mg/mL Na₂EDTA*2H₂O and pH 3.3-4.2.

9. Pharmaceutical composition according to claim 7, comprising 2.5-3.5 mM epinephrine, an antioxidant selected from the group consisting of 2.3-3 mM sodium metabisulfite, 4.6-6 mM sodium sulfite and 4.6-6 mM sodium bisulfite, 2.5-3.5 mM tartrate, 8.5-9.5 mg/mL NaCl, 0.09-0.16 mg/mL EDTA or 0.12-0.2 mg/mL Na₂EDTA*2H₂O and pH 3.3-4.2.

10. Process for the preparation of a pharmaceutical composition according to claim 1, characterized in that epinephrine, an antioxidant selected from the group consisting of sodium metabisulfite, sodium sulfite and sodium bisulfite, NaCl and EDTA or Na₂EDTA*2H₂O together with water are brought into a suitable dosage form.

11. Pharmaceutical composition according to claim 1, for use in the treatment of physiological and/or pathophysiological states, selected from the group consisting allergic reactions in the context of allergic emergencies and anaphylaxis and anaphylactoid reactions in the context of systemic toxic responses.

12. Glass syringe comprising a pharmaceutical composition according to claim 1.

13. Auto-injector comprising a pharmaceutical composition according to claim 1.

14. Set (kit) consisting of separate packs of
a) a glass syringe or an auto-injector and
b) a pharmaceutical composition according to claim 1, stored in a glass container, ampoule, prefilled cartridge or vial.
